SEQUENCE LISTING (4 sequences)

SEQ ID NO: 1 - protein, 395 aa, synthetic construct
  source 1..395: note = recombinant human acid ceramidase
MPGRSCVALVLLAAAVSCAVAQHAPPWTEDCRKSTYPPSGPTYRGAVPWYTINLDLPPYKRWHELMLDKAPVLKVIVNSLKNMINTFVPSGKIMQVVDEKLPGLLGNFPGPFEEEMKGIAAVTDIPLGEIISFNIFYELFTICTSIVAEDKKGHLIHGRNMDFGVFLGWNINNDTWVITEQLKPLTVNLDFQRNNKTVFKASSFAGYVGMLTGFKPGLFSLTLNERFSINGGYLGILEWILGKKDVMWIGFLTRTVLENSTSYEEAKNLLTKTKILAPAYFILGGNQSGEGCVITRDRKESLDVYELDAKQGRWYVVQTNYDRWKHPFFLDDRRTPAKMCLNRTSQENISFETMYDVLSTKPVLNKLTVYTTLIDVTKGQFETYLRDCPDPCIGW

SEQ ID NO: 2 - DNA, 2618 bp, synthetic construct
  source 1..2618: mol_type = other DNA; note = encodes recombinant human acid ceramidase
ggctcggtccgactattgcccgcggtgggggagggggatggatcacgccacgcgccaaaggcgatcgcgactctccttctgcaggtagcctggaaggctctctctctttctctacgccacccttttcgtggcactgaaaagccccgtcctctcctcccagtcccgcctcctccgagcgttccccctactgcctggaatggtgcggtcccaggtcgcgggtcacgcggcggagggggcgtggcctgcccccggcccagccggctcttctttgcctctgctggagtccggggagtggcgttggctgctagagcgatgccgggccggagttgcgtcgccttagtcctcctggctgccgccgtcagctgtgccgtcgcgcagcacgcgccgccgtggacagaggactgcagaaaatcaacctatcctccttcaggaccaacgtacagaggtgcagttccatggtacaccataaatcttgacttaccaccctacaaaagatggcatgaattgatgcttgacaaggcaccagtgctaaaggttatagtgaattctctgaagaatatgataaatacattcgtgccaagtggaaaaattatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtgagcacacgtctggcctacagaatgcggcctctgagacatgaagacaccatctccatgtgaccgaacactgcagctgtctgaccttccaaagactaagactcgcggcaggttctctttgagtcaatagcttgtcttcgtccatctgttgacaaatgacagatcttttttttttccccctatcagttgatttttcttatttacagataacttctttaggggaagtaaaacagtcatctagaattcactgagttttgtttcactttgacatttggggatctggtgggcagtcgaaccatggtgaactccacctccgtggaataaatggagattcagcgtgggtgttgaatccagcacgtctgtgtgagtaacgggacagtaaacactccacattcttcagtttttcacttctacctacatatttgtatgtttttctgtataacagccttttccttctggttctaactgctgttaaaattaatatatcattatctttgctgttattgacagcgatataattttattacatatgattagagggatgagacagacattcacctgtatatttcttttaatgggcacaaaatgggcccttgcctctaaatagcactttttggggttcaagaagtaatcagtatgcaaagcaatcttttatacaataattgaagtgttccctttttcataattactctacttcccagtaaccctaaggaagttgctaacttaaaaaactgcatcccacgttctgttaatttagtaaataaacaagtcaaagacttgtggaaaataggaagtgaacccatattttaaattctcataagtagcattcatgtaataaacaggtttttagtttgttcttcagattgatagggagttttaaagaaattttagtagttactaaaattatgttactgtatttttcagaaatcaaactgcttatgaaaagtactaatagaacttgttaacctttctaaccttcacgattaactgtgaaatgtacgtcatttgtgcaagaccgtttgtccacttcattttgtataatcacagttgtgttcctgacactcaataaacagtcactggaaagagtgccagtcagcagtcatgcacgctgattgggtgtgt

SEQ ID NO: 3 - DNA, 1276 bp, synthetic construct
  source 1..1276: mol_type = other DNA; note = encodes recombinant human acid ceramidase
aagcttaccgccaccatgaactgctgcatcggcctgggtgagaaggcgcgtggctcgcaccgcgccagctacccctccctgagcgccctcttcaccgaggcgtccatcctcggattcgggagcttcgccgtcaaggcacagtggaccgaggattgccgcaagagtacgtacccccccagtggcccgacgtaccgcggcgccgtcccctggtacacgatcaacctggacctccccccgtacaagcgctggcacgagttgatgctggacaaggcccccgtactgaaggtcatcgtgaactccctgaagaacatgatcaacaccttcgtcccctcgggcaagatcatgcaggtcgtggacgagaagctgcccgggctcctcggcaacttccccggcccgttcgaagaggagatgaagggcatcgcggccgtcactgacatccccctgggcgagatcatcagcttcaacatcttctacgagctgttcaccatctgcacctccatcgtagccgaggacaagaagggccacctgatccacggtcgcaacatggacttcggcgtcttcctgggctggaacatcaacaacgacacctgggtcatcaccgagcagctgaagccgctcaccgtgaacctcgatttccagcgcaacaacaagacggtgttcaaggccagctccttcgccgggtacgtcgggatgctcacgggcttcaagccgggactgttctcgctgaccctcaacgagcggttctccatcaacgggggctacctcggcatcctggagtggattctcggcaagaaggacgtgatgtggatcggcttcctcacacggaccgtgctggaaaactccactagttacgaggaggccaagaacctgctgaccaagacgaagatcctggccccggcatacttcatcctgggcggcaaccagtcgggcgaggggtgcgtcatcacccgcgaccggaaggagtccctggacgtctatgagctggacgccaagcagggccgctggtacgtcgtccagacgaactacgaccgatggaagcaccccttcttcctcgacgaccggcgcacgcccgccaagatgtgcctgaaccgcaccagccaggagaacatctcgttcgagacgatgtacgacgtgctgtcgaccaagcccgtgctcaacaagctgacggtctacaccacgctgatcgacgtgacgaaaggccagttcgaaacgtacctgcgggactgcccggacccttgcatcggctggtgataatctagagtcggggcggccggcc

SEQ ID NO: 4 - DNA, 1276 bp, synthetic construct
  source 1..1276: mol_type = other DNA; note = encodes recombinant human acid ceramidase
aagcttaccgccaccatgaactgctgcatcgggctgggagagaaagctcgcgggtcccaccgggcctcctacccaagtctcagcgcgcttttcaccgaggcctcaattctgggatttggcagctttgctgtgaaagcccaatggacagaggactgcagaaaatcaacctatcctccttcaggaccaacgtacagaggtgcagttccatggtacaccataaatcttgacttaccaccctacaaaagatggcatgaattgatgcttgacaaggcaccagtgctaaaggttatagtgaattctctgaagaatatgataaatacattcgtgccaagtggaaaaattatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttccagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtgataacctagggtcggggcggccggcc

(19) United States
(12) Patent Application Publication
(10) Pub. No.: US 2026/0000741 A1
(43) Pub. Date: Jan. 1, 2026

(54) METHOD OF AMELIORATING A PRO-INFLAMMATORY IMMUNOPHENOTYPE IN FARBER DISEASE SUBJECTS BY REPEATED ADMINISTRATION OF A RECOMBINANT HUMAN ACID CERAMIDASE

(71) Applicant: ACERAGEN, INC., Research Triangle Park, NC (US)

(72) Inventors: Brante P. SAMPEY, Durham, NC (US); Christine M. COQUERY, Durham, NC (US); Alexander Istvan SOLYOM, Freiburg im Breisgau (BA)

(21) Appl. No.: 19/054,448

(22) Filed: Feb. 14, 2025

Related U.S. Application Data
(63) Continuation of application No. 17/425,207, filed on Jul. 22, 2021, now abandoned.
(60) Provisional application No. 62/795,621, filed on Jan. 23, 2019.

(51) Int. Cl. A61K 38/50; A61K 45/06; A61P 37/06

(52) U.S. Cl. CPC A61K 38/50; A61K 45/06; A61P 37/06

(57) ABSTRACT

Compositions and methods for treating inflammation associated with Farber disease in a subject in need thereof by administering to the subject a pharmaceutical composition comprising a recombinant human acid ceramidase in a therapeutically effective amount of about 0.1 mg/kg to about 50 mg/kg to inhibit inflammation and/or to inhibit or reduce pro-inflammatory potential of neutrophils and/or monocytes in the subject.

### CROSS REFERENCE TO RELATED APPLICATION

This application claims the benefit to and priority of U.S. Provisional Application No. 62/795,621 filed on Jan. 23, 2019, the subject matter of which is incorporated herein by reference.

### FIELD OF INVENTION

Compositions and methods for treating pro-inflammatory immunophenotypes in Farber disease through repeated administration of a recombinant human acid ceramidase.

### BACKGROUND OF THE INVENTION

Farber disease (“FD”) is an ultra-rare lysosomal storage disorder, with an estimated worldwide prevalence of < 1/1,000,000. Farber disease was first described by Dr. Sidney Farber in 1952. The disease was first identified in a 14-month-old infant with granulomatous lesions on multiple joints and concomitant evidence of lipid storage. Farber disease typically presents early in childhood. Joint contractures, subcutaneous nodules, and progressive hoarseness of voice are frequently observed in children afflicted with Farber disease. (Farber, S. (1952) A lipid metabolic disorder-disseminated “Lipogranulomatosis”—a syndrome with similarity to, and important difference from, Niemann-Pick and Hand-Schuller-Christian disease,” Am. J. Dis. Child., 84:49).

Following the identification of the principal symptoms of Farber Disease in 1952, similar cases were thereafter described in the ensuing decade. These individuals all had similar lesions and often exhibited a characteristic “hoarse” voice resulting from lesions on the larynx. The involvement of other organ systems in some of these patients, including the lung, liver, spleen and central nervous system (“CNS”), also was observed.

A broad phenotypic variance is observed in subjects with Farber disease. The phenotypic variance may be related to the specific acid ceramidase (“AC”) mutation that has occurred in the subject.

Farber disease is an ultra-rare autosomal recessive lysosomal storage disease associated with gene mutations of the ASAH1 gene. Such mutations result in non- or minimally functional AC, leading to accumulation of pro-inflammatory ceramide in tissues accompanied by severe inflammation. This phenotypic variance largely determines the progression of the disease.

Deficiency in AC enzyme function results in accumulation of sphingolipids. This results in painful abnormalities, such as painful nodules, in the joints, liver, throat, and various tissues, as well as central nervous system (CNS) symptoms. Mutations in the AC enzyme have been identified in patients with Farber disease (Koch et al., 1996, Molecular Cloning and Characterization of a Full-Length Complementary DNA Encoding Human Acid Ceramidase, J. Biol. Chem. 271:33110-33115). Currently, non-disease specific treatments available for Farber disease include pain management with anti-inflammatory medications including corticosteroids, anti-TNFα and anti-IL-6 antibody therapies, and bone marrow (or hematopoietic stem cell) transplantation. Enzyme replacement therapy with recombinant human AC (“rhAC”) has been proposed as an alternative to bone marrow transplantation. (Solyom A, Huegle B, Magnusson B, Makay B, Arslan N, Mitchell J, Tanpaiboon P, Guelbert N, Puri R, Jung L, Grigelioniene G, Ehlert K, Beck M, Simonaro C, Schuchman E. “Farber Disease: Important Differential Diagnostic Information for JIA and Other Inflammatory Arthritis Phenotypes Is Revealed By Data from the Largest Clinical Cohort to Date,” Arthritis Rheumatol., 2015:67 (sup. 10), abstract.

Severely affected individuals often succumb to respiratory complications or hepatic failure within the first two years of life and may have profoundly delayed mental development and cognitive impairment. Patients with milder phenotypes can, however, live well into adulthood.

Due to the characteristic inflammation of articular tissues and joint pathologies in pediatric patients, Farber disease may be misdiagnosed as juvenile idiopathic arthritis (Lampe C, Bellettato C, Karabul N, and Scarpa M., 2013, Mucopolysaccharidoses and other lysosomal storage diseases. Rheum Dis Clin N Am. May; (39) (2): 431-55: Hugle B, Mueller L, Levade T., 2014. “Why Farber disease may be misdiagnosed as juvenile idiopathic arthritis,” The Rheumatologist, June 1)

Normally, acid ceramidase (also referred to as “N-acylsphingosine deacylase”) is taken up intracellularly into lysosomes where it hydrolyzes ceramide to sphingosine and free fatty acids at lysosomal pH.

In Farber disease (“FD”), the non-functional AC is incapable of reducing its substrate, and ceramide accumulate across a wide range of tissues, leading to inflammation and macrophage infiltration. The formation of large depots of histiocytes (also referred to as “activated tissue-resident macrophages”) contributes to disease progression and pathology.

Previously, treatment for Farber disease patients has been symptomatic and principally aimed at reducing pain. Hematopoietic stem cell transplantation (HSCT) has been undertaken in a limited number of patients, and overall the outcome has been positive provided that the transplant procedure itself was successful. Such transplanted patients exhibit significant reduction in pain, increased motility and mobility, and in some cases size reduction or complete resolution of the subcutaneous nodules. Successful transplantation, however, requires histocompatible donor cells, and exposes patients to invasive and potentially dangerous immunosuppressant regimes.

One alternative to HSCT is gene therapy in which autologous donor cells are transduced with a vector expressing the therapeutic protein, obviating the need for histocompatible donors. This approach has been evaluated in the Farber disease knock-in mouse model and resulted in reduction of tissue ceramide and macrophage infiltration.

Treatment of Farber disease mice with a recombinant human acid ceramidase has demonstrated reduced ceramide content in multiple tissues and lowered monocyte chemoattractant protein (MCP)-1. Although upregulation of plasma monocyte chemoattractant protein (MCP)-1 and histiocytic infiltration of tissues has been described in Farber disease, the inflammatory cells contributing to disease pathology have not been well characterized. (Dworski, S., et al., 2017, “Acid ceramidase deficiency is characterized by a unique plasma cytokine and ceramide profile that is altered by therapy.” S. Dworski et al. Biochimica et Biophysica Acta 1863 (2017) 386-394): See also Schuchman, E. (inventor), Icahn School of Medicine at Mount Sinai (applicant), 2018 Jan. 12, Compositions and Methods for Treating Farber Disease, International application under the Patent Cooperation Treaty, WO 2018/132667, which is hereby incorporated by reference in its entirety).

Previously, it has been reported that acid ceramidase enzyme replacement therapy (“ERT”) reduced spleen weight in the Farber disease mice to that of normal mice, even at a 1 mg/kg dose. Plasma MCP-1, a macrophage chemokine that is elevated in Farber disease mice and also was significantly reduced by ERT, although the levels never reached those of normal animals patients (Dworski et al., 2017).

The previous findings suggested that ERT in Farber disease mice had a significant impact on several important endpoints, including tissue ceramide levels, sphingosine levels, macrophage infiltration, spleen size, and plasma MCP-1 levels. There also was some evidence that rhAC might influence the brain (e.g., reduction of accumulating ceramide and restoration of suppressed sphingosine).

Consistent with the reduction in MCP-1, reduced macrophage infiltration into Farber disease mouse liver and spleen tissue following recombinant acid ceramidase administration was observed previously.

There, nevertheless, continues to be a need for improved therapies for treating Farber disease. The present subject matter fulfills these needs as well as others as described below.

### SUMMARY OF THE INVENTION

In accordance with the description, figures, examples and claims of the present specification, the inventors have demonstrated a novel, inventive and efficacious treatment of Farber disease.

Using a Farber disease mouse model, flow cytometry was utilized to characterize the immune cell repertoire in blood and key tissues (lung, liver, spleen). Farber disease mice homozygous for the Asah1^{P361R/P361R} mutation, (hom) were utilized in all experiments. Compared to wild-type (WT) mice, multiple pro-inflammatory immune cells were broadly elevated in Farber disease mice, including neutrophils (2-7-fold increase), activated monocytes (3-6-fold increase) and activated B cells (2-fold increase). In contrast, total T cells were decreased in the blood and all Farber disease mouse tissues examined. Changes in immune cell composition were observed in Farber mice as young as 4 weeks and remained comparable or slightly exacerbated with increasing age.

To assess whether recombinant human acid ceramidase directly impacted the immune cell compartment, Farber disease mice were treated with four once-weekly intraperitoneal doses at 10 mg/kg/dose—the maximally effective dose in the Farber mouse model. Recombinant human acid ceramidase decreased pro-inflammatory immune cells, with key reductions in neutrophils and activated monocytes to wild-type control levels in the lung, liver, blood, and to a lesser extent the spleen.

These findings, together with the previously reported data showing MCP-1 and ceramide reduction, demonstrate amelioration of the pro-inflammatory status of the Farber disease mouse model following repeat dose treatment with recombinant human acid ceramidase and further support its potential as an enzyme replacement therapy for Farber disease. Moreover, the study identifies potential biomarkers that may be relevant to Farber disease patients and in response to treatment with rhAC.

Across tissue samples that were collected, neutrophils are rapidly increased and maintained in the Asah1^{P361R/P361R} Farber mouse model.

Secondary to an increase in activated monocytes is a switching from protective M2 to an inflammatory M1 phenotype, which may be dependent on early cytokine/chemokine signals that are neutrophil derived.

Heat map changes show a discrete peripheral immune profile emerging in Farber mice reflecting changes that are observed in the tissues. In all instances, Farber mice treated with recombinant human acid ceramidase (i.e., with “RVT-801”) show a marked decrease in activated inflammatory cell populations as measured by neutrophil and monocyte populations. While B-cells were elevated, the main response related to dosing with rhAC was upon neutrophils and activated monocytes and macrophages. While changes in T cells were also noted in Farber mice and wild type mice, drug related changes could not be detected.

Taken together, the experimental findings demonstrate that in addition to an early increase in ceramide, inflammation likely contributes directly to the Farber phenotype, and recombinant human acid ceramidase administration (administered as RVT-801) suppresses an inflammatory environment and, therefore, should be useful as an enzyme replacement therapy for the treatment of Farber disease.

In a first aspect of the invention disclosed herein, a therapeutically effective amount of a recombinant human acid ceramidase (rhAC) is administered to a subject in need thereof to reduce inflammation associated with Farber disease.

A second aspect of the invention disclosed herein, is a method for treating inflammation in tissues and organs of a subject in need thereof having Farber disease, comprising administering to the subject a therapeutically effective amount of a recombinant human acid ceramidase to inhibit or reduce a pro-inflammatory potential of neutrophils and/or monocytes in the subject.

In an embodiment of the first aspect, the method for treating inflammation associated with Farber disease in a subject in need thereof, the method comprising administering to the subject a pharmaceutical composition comprising a recombinant human acid ceramidase in a therapeutically effective amount of about 0.1 mg/kg to about 50 mg/kg.

In an embodiment of the second aspect, the method is for treating inflammation in tissues and organs of a subject in need thereof having Farber disease, comprising administering to the subject a therapeutically effective amount of a recombinant human acid ceramidase to inhibit or reduce pro-inflammatory potential of neutrophils and/or monocytes in the subject.

In an embodiment of the first aspect recombinant human acid ceramidase (e.g., RVT-801) is repeatedly administered in a pharmaceutically acceptable composition to a subject in need thereof to reduce inflammation associated with Farber disease to reduce ceramide levels and to suppress a pro-inflammatory environment present in subjects having Farber disease.

In some embodiments disclosed herein, the methods comprise administering to a subject in need thereof a pharmaceutical composition comprising a human recombinant acid ceramidase in an effective amount of about 0.1 mg/kg to about 50 mg/kg to reduce ceramide levels in a subject and to reduce inflammation associated with Farber disease. Reducing ceramide can also refer to decreasing ceramide or increasing the metabolizing of ceramide, which would lead to reduced ceramide levels. In some embodiments, the human recombinant acid ceramidase is RVT-801.

In some embodiments the pharmaceutical composition comprises a human recombinant acid ceramidase in an effective amount of about 1 mg/kg to about 10 mg/kg and is administered to a subject to reduce inflammation associated with Farber disease. In some embodiments, the human recombinant acid ceramidase is RVT-801.

In some embodiments the pharmaceutical composition comprises a human recombinant acid ceramidase in an effective amount of about 1 mg/kg to about 5 mg/kg and is administered to a subject to reduce inflammation associated with Farber disease. In some embodiments, the human recombinant acid ceramidase is RVT-801.

In some embodiments disclosed herein, methods of reducing ceramide levels in a subject with, or suspected of having, Farber disease are provided. In some embodiments, the subject is a subject in need thereof. In some embodiments, the methods comprise administering to the subject a pharmaceutical composition comprising a human recombinant acid ceramidase in an effective amount of about 0.1 mg/kg to about 50 mg/kg to reduce ceramide level in tissues affected by Farber disease and to reduce neutrophil and/or monocyte populations of pro-inflammatory cells associated with Farber disease. In some embodiments, the human recombinant acid ceramidase is RVT-801. Reducing ceramide can also refer to decreasing ceramide or increasing the metabolizing of ceramide, which would lead to reduced ceramide levels.

In some embodiments the pharmaceutical composition comprises a human recombinant acid ceramidase in an effective amount of about 1 mg/kg to about 10 mg/kg and is administered to a subject to reduce ceramide level in tissues affected by Farber disease and to reduce neutrophil and/or monocyte populations of pro-inflammatory cells associated with Farber disease. In some embodiments, the human recombinant acid ceramidase is RVT-801.

In some embodiments the pharmaceutical composition comprises a human recombinant acid ceramidase in an effective amount of about 1 mg/kg to about 5 mg/kg and is administered to a subject to reduce ceramide level in tissues affected by Farber disease and to reduce neutrophil and/or monocyte populations of pro-inflammatory cells associated with Farber disease. In some embodiments, the human recombinant acid ceramidase is RVT-801.

In an embodiment of the second aspect, recombinant human acid ceramidase (e.g., RVT-801) is repeatedly administered in a pharmaceutically acceptable composition to a subject in need thereof to reduce neutrophil and/or monocyte populations of pro-inflammatory cells associated with Farber disease.

A third aspect of the present invention comprises administering to a subject in need thereof a pharmaceutical composition comprising a human recombinant acid ceramidase in an effective amount of about 0.1 mg/kg to about 50 mg/kg to increase sphingosine levels and to reduce inflammation associated with Farber disease. In some embodiments, the human recombinant acid ceramidase is RVT-801.

In some embodiments the pharmaceutical composition comprises a human recombinant acid ceramidase in an effective amount of about 1 mg/kg to about 10 mg/kg and is administered to a subject to increase sphingosine levels and to reduce inflammation associated with Farber disease. In some embodiments, the human recombinant acid ceramidase is RVT-801.

In some embodiments the pharmaceutical composition comprises a human recombinant acid ceramidase in an effective amount of about 1 mg/kg to about 5 mg/kg and is administered to a subject to increase sphingosine levels and to reduce inflammation associated with Farber disease. In some embodiments, the human recombinant acid ceramidase is RVT-801.

A fourth aspect of the present invention comprises administering to a subject in need thereof a human recombinant acid ceramidase in an effective amount of about 0.1 mg/kg to about 50 mg/kg to increase sphingosine levels and to reduce neutrophil and/or monocyte populations of pro-inflammatory cells associated with Farber disease. In some embodiments, the human recombinant acid ceramidase is RVT-801.

In some embodiments the pharmaceutical composition comprises a human recombinant acid ceramidase in an effective amount of about 1 mg/kg to about 10 mg/kg and is administered to a subject to increase sphingosine levels and to reduce neutrophil and/or monocyte populations of pro-inflammatory cells associated with Farber disease. In some embodiments, the human recombinant acid ceramidase is RVT-801.

In some embodiments the pharmaceutical composition comprises a human recombinant acid ceramidase in an effective amount of about 1 mg/kg to about 5 mg/kg and is administered to a subject to increase sphingosine levels and to reduce neutrophil and/or monocyte populations of pro-inflammatory cells associated with Farber disease. In some embodiments, the human recombinant acid ceramidase is RVT-801.

A fifth aspect of the present invention comprises administering to a subject in need thereof a pharmaceutical composition comprising a human recombinant acid ceramidase in an effective amount of about 0.1 mg/kg to about 50 mg/kg to increase sphingosine levels and reduce ceramide levels in tissues affected by Farber disease and to reduce neutrophil and/or monocyte populations of pro-inflammatory cells associated with Farber disease. In some embodiments, the human recombinant acid ceramidase is RVT-801.

In some embodiments the pharmaceutical composition comprises a human recombinant acid ceramidase in an effective amount of about 1 mg/kg to about 10 mg/kg and is administered to a subject to increase sphingosine levels and reduce ceramide levels in tissues affected by Farber disease and to reduce neutrophil and/or monocyte populations of pro-inflammatory cells associated with Farber disease. In some embodiments, the human recombinant acid ceramidase is RVT-801.

In some embodiments the pharmaceutical composition comprises a human recombinant acid ceramidase in an effective amount of about 1 mg/kg to about 5 mg/kg and is administered to a subject to increase sphingosine levels and reduce ceramide levels in tissues affected by Farber disease and to reduce neutrophil and/or monocyte populations of pro-inflammatory cells associated with Farber disease. In some embodiments, the human recombinant acid ceramidase is RVT-801.

In an embodiment of the foregoing aspects of the invention, the acid ceramidase comprises UniProt Q13510, UniProt Q9H715, UniProt Q96AS2, OMIM 228000, NCBI Gene 427, NCBI RefSeq NP_808592, NCBI RefSeq NP_004306, NCBI RefSeq NM_177924, NCBI RefSeq NM_004315, NCBI UniGene 427, NCBI Accession Q13510, NCBI Accession AAC73009, or a combination thereof.

In an embodiment of the foregoing aspects of the invention, the recombinant human acid ceramidase is a rhAC encoded by the ASAH1 gene (NCBI UniGene GeneID No. 427).

In an embodiment of the foregoing aspects of the invention, the recombinant human acid ceramidase comprises the sequence of SEQ ID NO: 1.

In an embodiment of the foregoing aspects of the invention, the recombinant human acid ceramidase comprises the sequence of UniProt Q13510.

In an embodiment of the foregoing aspects of the invention, the recombinant human acid ceramidase comprises the sequence of NCBI RefSeq NP_808592.

In a sixth aspect of the present invention disclosed herein, a therapeutically effective amount of a recombinant human acid ceramidase (rhAC) and a second anti-inflammatory agent is administered to a subject in need thereof to reduce inflammation associated with Farber disease.

In an embodiment of the sixth aspect of the present invention, the second anti-inflammatory agent is selected from the group consisting of aceclofenac, alclofenac, amfenac, aminophenazone, ampiroxicam, ampyrone, amtolmetin guacil, anitrazafen azapropazone, bendazac, benzydamine, bromfenac, bumadizone, carprofen, celecoxib, cimicoxib, clofezone, clonixin, copper ibuprofenate, COX-inhibiting nitric oxide donator, deracoxib, dexibuprofen, dexketoprofen, diclofenac, diclofenac/misoprostol, diflunisal, droxicam, epirizole, ethenzamide, etodolac, etofenamate, etoricoxib, famprofazone, felbinac, fenamic acid, fenbufen, fenclofenac, fenclozic acid, fenoprofen, feprazone, firocoxib, floctafenine, flumizole, flunixin, fluproquazone, flurbiprofen, ibuprofen, indomethacin, indometacin farnesil, indoprofen, ketoprofen, ketorolac, licofelone, lonazolac, lornoxicam, loxoprofen, lumiracoxib, magnesium salicylate, mavacoxib, mefenamic acid, meloxicam, meseclazone, miroprofen, mofebutazone, morazone, nabumetone, naproxcinod, naproxen, nepafenac, nimesulide, NOSH-aspirin, NS-398, oxaprozin, oxicam, oxyphenbutazone, parecoxib, phenazone, phenylbutazone, piroxicam, pirprofen, pranoprofen, proglumetacin, robenacoxib, rofecoxib, salicylic acid, salsalate, sulindac, suprofen, tarenflurbil, tenidap, tenoxicam, tepoxalin, tiaprofenic acid, tocilizumab, tolfenamic acid, tolmetin, valdecoxib, vedaprofen, and zomepirac.

In an embodiment of the foregoing first to sixth aspects of the invention, the pharmaceutical composition comprises about 0.1 mg/kg to about 50 mg/kg of recombinant human acid ceramidase.

In an embodiment of the foregoing first to sixth aspects of the invention, the pharmaceutical composition comprises about 1 mg/kg to about 10 mg/kg of recombinant human acid ceramidase.

In an embodiment of the foregoing first to sixth aspects of the invention, the pharmaceutical composition comprises about 1 mg/kg to about 5 mg/kg of recombinant human acid ceramidase.

In an embodiment of the foregoing first to sixth aspects of the invention, the pharmaceutical composition is in solid or liquid form.

In an embodiment of the foregoing first to sixth aspects of the invention, the pharmaceutical composition comprises RVT-801.

In an embodiment of the foregoing first to sixth aspects of the invention, the liquid form of the pharmaceutical composition is a sterile injectable solution.

In an embodiment of the foregoing first to sixth aspects of the invention, the liquid form of the pharmaceutical composition is a sterile dispersion.

In an embodiment of the foregoing first to sixth aspects of the invention, the pharmaceutical composition is in the form selected from the group consisting of tablets, capsules, elixirs, suspensions, a solution, a dispersion (including aerosol and dry powder inhalants), and syrups.

In an embodiment of the foregoing first to sixth aspects of the invention, the pharmaceutical composition further comprises one or more of the following: a binder, an excipient, a disintegrating agent, a lubricant, a sweetening agent, or a liquid carrier.

In an embodiment of the foregoing first to sixth aspects of the invention, the pharmaceutical composition comprises saline or water.

Various pharmaceutical compositions are described herein and can be used based upon the patients' and doctors' preferences. In some embodiments, the pharmaceutical composition is a solution.

In some embodiments, the pharmaceutical composition comprises cell conditioned media comprising the recombinant human acid ceramidase. As used herein, the term “cell conditioned media” refers to cell culture media that has been used to culture cells expressing recombinant human acid ceramidase and where the protein is secreted into the media and then the protein is isolated or purified from the media.

In some embodiments, methods of treating Farber disease in a subject in need thereof are provided, wherein the method comprises expressing recombinant human acid ceramidase in a cell; isolating the expressed rhAC from the cell; and administering to the subject a pharmaceutical composition comprising the isolated expressed recombinant human acid ceramidase in an therapeutically effective amount of about 0.1 mg/kg to about 50 mg/kg.

### BRIEF DESCRIPTION OF THE DRAWINGS

For a more complete understanding of the principles disclosed herein, and the advantages thereof, reference is made to the following descriptions taken in conjunction with the accompanying drawings, in which:

FIGS. 1A-E show a representative immunophenotyping gating strategy of mouse splenocytes in a Farber “knock-in” mouse treated with recombinant human acid ceramidase (RVT-801), as described in Example 1.

FIGS. 2A-C show splenic immune cell populations in wild-type (WT) mice, a Farber “knock-in” mouse treated with vehicle (saline), or a Farber mouse treated with repeat doses of recombinant human acid ceramidase (RVT-801), Splenic immune cell populations are elevated in control Farber mice when compared to WT splenic immune cell populations, and are decreased in Farber mice treated with recombinant human acid ceramidase (RVT-801), as described in Example 2.

FIGS. 3A-C show systemic immune cell populations in WT mice, a Farber “knock-in” mouse treated with vehicle (saline) or a Farber mouse treated with repeat doses of recombinant human acid ceramidase (RVT-801). Systemic immune cell populations are elevated in control Farber mice when compared to WT systemic immune cell populations, and are decreased in Farber mice treated with recombinant human acid ceramidase (RVT-801), as described in Example 3.

FIGS. 4A-D show pulmonary immune cell populations in WT mice, a Farber “knock-in” mouse treated with vehicle (saline), or a Farber mouse treated with repeat doses of recombinant human acid ceramidase (RVT-801), Lung immune cell populations are elevated in control Farber mice when compared to WT lung immune cell populations, and are decreased in Farber mice treated with recombinant human acid ceramidase (RVT-801), as described in Example 4.

FIG. 5A-B show hepatic immune cell populations in WT mice, in a Farber “knock-in” mouse treated with saline), or a Farber mouse treated with repeat doses of recombinant human acid ceramidase (RVT-801), Liver immune cell populations are elevated in control Farber mice when compared to WT liver immune cell populations, and are decreased in Farber mice treated with recombinant human acid ceramidase (RVT-801), as described in Example 5.

FIGS. 6A-O show hematoxylin and eosin (H&E) stained bone specimens from WT mice, a Farber mice treated with vehicle (saline), or a Farber (Asah1^{P361R/P361R}) mouse treated with repeat doses of recombinant human acid ceramidase (RVT-801). In control Farber mouse, thinning of the physis and bony trabeculae, replacement of fat pads with histiocyte infiltrates and histiocyte infiltration of the bone marrow were observed in comparison to the WT joint and bone. Farber mice were administered recombinant human acid ceramidase (RVT-801) at a dose of 0, 0.1, 1, 3 or 10 mg/kg once-weekly starting at three weeks of age for 6 weeks showed reduced thinning of the physis and bony trabeculae and amelioration of joint fat pads with histiocyte infiltrates. Importantly, histiocyte infiltration of the bone marrow that was observed in control Farber mice was not present in Farber mice treated with 10 mg/kg/dose RVT-801 once-weekly for 6 weeks; not different that WT histology.

FIG. 7 shows in systemic blood specimens a significant elevation in the inflammatory cytokine monocyte chemoattractant protein (MCP-1) in control Farber mice when compared to wild type mice, and a dose related decrease in systemic MCP-1 in Farber mice treated with recombinant human acid ceramidase (RVT-801) at concentrations of 0.1, 1, 3, or 10 mg/kg/dose once-weekly for 6 weeks. Eight Farber mice were administered recombinant human acid ceramidase (RVT-801) at a dose of 0, 0.1, 1, 3 and 10 mg/kg weekly starting at three weeks of age.

FIG. 8A shows foamy histiocyte infiltrates and reduced red and white pulp in spleens of Farber mice treated with RVT-801 compared to wild type and Farber mice. Images are shown at 4× and 20× magnification. FIG. 8B shows a dose-dependent reduction in spleen weight in Farber mice treated with RVT-801. FIG. 8C shows a dose-dependent reduction in ceramide levels in spleens of Farber mice dosed with 0, 0.1, 1, 3 and 10 mg/kg RVT-801 compared to untreated mice. FIG. 8D shows increased sphingosine in a dose dependent manner upon administration of RVT-801 compared to wild type mice.

FIG. 9A shows histological evaluation of liver specimens for wild type, control Farber mice and Farber mice treated with RVT-801. Histiocyte infiltration of the liver and histiocyte-related vascular thrombi were observed in control Farber mice when compared to WT livers. Treatment of Farber mice with increasing RVT-801 ameliorated liver histiocytic infiltration and vascular thrombi to levels no different that WT controls. FIGS. 9B and 9C show a dose-dependent reduction in ceramide levels in livers of Farber mice dosed with 0, 0.1, 1, 3 and 10 mg/kg RVT-801 compared to untreated mice, nearly approaching levels in wild type mice.

FIGS. 10A-C show images of lungs of WT mice, control Farber mice and Farber mice treated with RVT-801. Control Farber mice show decreased aveolar sac patency, alveolar wall thickening, proteinosis (proteinaceous filling of the alveolar sacs), concomitant with increased histiocytic infiltration of the lung when compared to WT lung. RVT-801 appeared to have minimal impact on the lung histopathology of Farber mice. FIG. 10B shows a comparison of lung to body weights in wild type mice, control Farber mice and Farber mice treated with RVT-801. RVT-801 somewhat decreased lung to body weight ratios in Faber mice. FIG. 10C shows little to no effect on ceramide levels in lungs of Farber mice treated with increasing doses of RVT-801, whereas FIG. 10D shows a dose-related increase in lung sphingosine levels in Farber mice treated with increasing doses of RVT-801, comparable to level noted in wild-type mice at the 10 mg/kg dose.

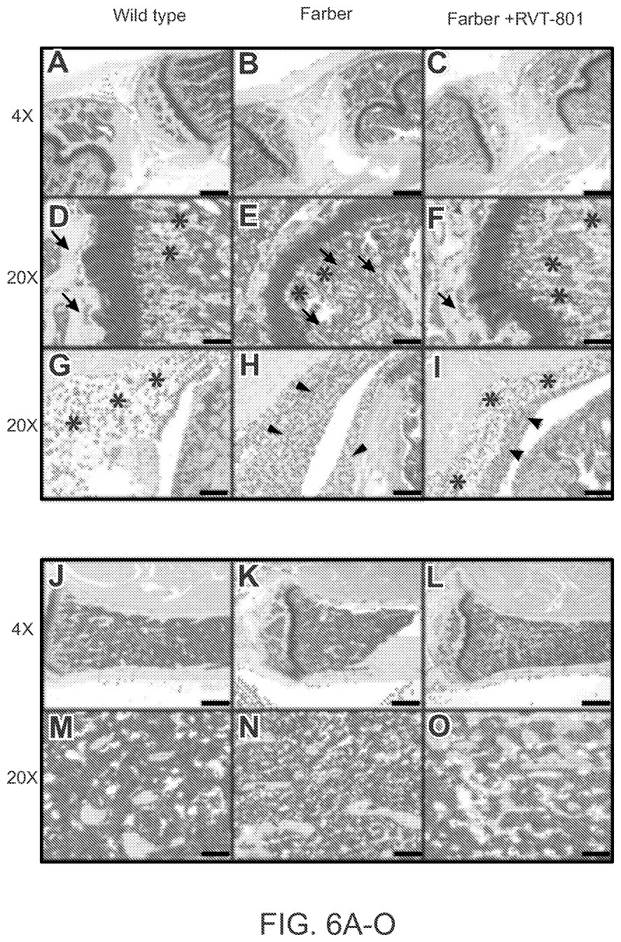

Data on brains of wild type mice, control Farber mice and Farber mice treated with RVT-801 is presented in FIGS. 11A-C. Glial necrosis occurred throughout the white matter of the forebrain, midbrain, and hindbrain in FM and was highly associated with areas of histiocytic infiltrates, which did not appear to be affected by RVT-801 treatment (FIG. 6A). FIG. 11B depicts untreated Farber mouse BW-normalized brain weights were approximately twice those of WT. Weekly RVT-801 doses of ≥3 mg/kg resulted in significantly decreased BW-normalized brain weights relative to untreated FM. Absolute Farber mouse brain weights were significantly lower than those of WT and generally decreased with dose and were not significantly different from WT absolute brain weight at ≥1 mg/kg/week (not shown). Farber mouse brains had significantly higher concentrations of ceramide, compared to WT, which decreased with RVT-801 administration but not in a dose-proportional manner (FIG. 11C). Conversely, RVT-801 treated Farber mouse brains contained lower concentrations of sphingosine than WT, levels that increased dose-dependently with increasing RVT-801 treatment, reaching WT levels at ≥3 mg/kg/dose (FIG. 11D).

### DETAILED DESCRIPTION OF THE INVENTION

The titles, headings and subheadings provided herein should not be interpreted as limiting the various aspects of the disclosure. Accordingly, the terms defined below are more fully defined by reference to the specification in its entirety. All references cited herein are incorporated by reference in their entirety.

Unless otherwise defined, scientific and technical terms used herein shall have the meanings that are commonly understood by those of ordinary skill in the art. Further, unless otherwise required by context, singular terms shall include pluralities and plural terms shall include the singular.

In this application, the use of “or” means “and/or” unless stated otherwise. In the context of a multiple dependent claim, the use of “or” refers back to more than one preceding independent or dependent claim in the alternative only.

It is further noted that, as used in this specification and the appended claims, the singular forms “a,” “an,” and “the,” and any singular use of any word, include plural referents unless expressly and unequivocally limited to one referent.

The instant invention is most clearly understood with reference to the following

### Definitions

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

As used herein, to “alleviate” a disease, defect, disorder or condition means reducing the severity of one or more symptoms of the disease, defect, disorder or condition.

As used herein, the term “animal” includes, but is not limited to, humans and non-human vertebrates such as wild, domestic, and farm animals. The animal can also be referred to as a “subject.”

As used herein, “anion exchange chromatography” or “AIEX” refers to a process that separates substances based on their charges using an ion-exchange resin containing positively charged groups. Capto Q is an exemplary AIEX media.

As used here, “biocompatible” refers to any material, which, when implanted in a mammal, does not provoke an adverse response in the mammal.

As used herein, the term “carrier” means a diluent, adjuvant, or excipient with which a compound is administered. Pharmaceutical carriers can be liquids, such as water and oils, including those of petroleum, animal, vegetable or synthetic origin, such as peanut oil, soy bean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

As used herein, “cation exchange chromatography” or “CIEX” refers to a process that separates substances based on their charges using an ion-exchange resin containing negatively charged groups. Capto S ImpAct is an exemplary CIEX media.

As used herein, “child” refers to an age that is from newborn to 18 years old.

As used herein, “chromatography” refers to any laboratory technique for separation of mixtures

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Additionally, a term that is used in conjunction with the term “comprising” is also understood to be able to be used in conjunction with the term “consisting of” or “consisting essentially of.”

As used herein, “ceramide accumulation inflammation” refers to inflammation caused by higher than normal levels of ceramide. Ceramide accumulation inflammation may occur based on changes in pH that result in an imbalance between ASM cleavage of sphingomyelin to ceramide and AC consumption of ceramide. Ceramide accumulation inflammation has been described in models of cystic fibrosis and may lead to pulmonary inflammation, respiratory epithelial cell death, DNA deposits in bronchi, and increased susceptibility to Pseudomonas aeruginosa infections (see Teichgräber, V. et al., 2008, Ceramide accumulation mediates inflammation, cell death and infection susceptibility in cystic fibrosis, Nature Medicine 14:382-391).

As used herein, the term “contacting” means bringing together of two elements in an in vitro system or an in vivo system. For example, “contacting” rhAC polypeptide an individual, subject, or cell includes the administration of the polypeptide to an individual or patient, such as a human, as well as, for example, introducing a compound into a sample containing a cellular or purified preparation containing the polypeptide. Additionally, contacting can refer to transfecting or infecting a cell with a nucleic acid molecule encoding the polypeptide.

An “effective amount” of an enzyme delivered to a subject is an amount sufficient to improve the clinical course of a Faber disease where clinical improvement is measured by any of the variety of defined parameters well known to the skilled artisan.

As used herein, the phrase “in need thereof” means that the subject has been identified as having a need for the particular method or treatment. In some embodiments, the identification can be by any means of diagnosis. In any of the methods and treatments described herein, the subject can be in need thereof.

As used herein, “Farber mouse,” “Farber mice,” “Farber disease mice,” and “Farber disease mouse” means severe Farber disease mouse model (Asah1^{P361R/P31R6}). The Farber mouse is a murine model based on a severe Farber disease patient genotype. Based on the severe Farber disease patient genotype, knock-in mice that are homozygous for a single nucleotide Asah1^{P361R/P361R} mutation were derived from a mixed genetic background colony (W4/129Sv/CD-1) (“CD-1 mice”) to establish a murine model of severe Farber disease, as previously described (Alayoubi, 2013). Farber mice have been established in knock-in mice homozygous for a single nucleotide mutation in the Asah1 gene. Farber (Asah1P361R/P31R6) mice produce an altered AC, incapable of hydrolyzing ceramides to their sphingosine and fatty acids constituents. These Farber mice exhibit features characteristic of clinical Farber disease including disruption of bone formation and the morphology of intra-articular tissues, presence of lipid-laden macrophages, and systemic inflammation, along with a significantly stunted rate of growth and shortened lifespan compared to their wild-type (Asah1WT/WT) or heterozygous (Asah1WT/P361R) littermates.

As used herein, “hydrophobic interaction chromatography” or “HIC” refers to a process that separates substances based on their hydrophobicity. Capto Butyl HIC is an exemplary HIC media.

As used herein, the term “include” and its grammatical variants are intended to be non-limiting, such that recitation of items in a list is not to the exclusion of other like items that can be substituted or added to the listed items.

The term “in combination with” as used herein means that the described agents can be administered to a subject together in a mixture, concurrently as single agents or sequentially as single agents in any order.

As used herein, the phrase “in need thereof” means that the subject has been identified as having a need for the particular method or treatment. In some embodiments, the identification can be by any means of diagnosis. In any of the methods and treatments described herein, the subject can be in need thereof.

As used herein, the phrase “integer from X to Y” means any integer that includes the endpoints. For example, the phrase “integer from 1 to 5” means 1, 2, 3, 4, or 5.

As used herein, the term “isolated” means that the compounds described herein are separated from other components of either (a) a natural source, such as a plant or cell, or (b) a synthetic organic chemical reaction mixture, such as by conventional techniques.

As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

As used herein, the term “purified” means that when isolated, the isolate contains at least 90%, at least 95%, at least 98%, or at least 99% of a compound described herein by weight of the isolate.

As used herein, “production,” “production process,” or “processing” refers to the production and purification of a biotherapeutic. Production can encompass bioengineering, equipment design, molecular biology, cell genetics, cell culture technology, and analytical chemistry.

As used herein, the phrase “substantially isolated” means a compound that is at least partially or substantially separated from the environment in which it is formed or detected.

As used herein, the terms “prevent”, “preventing” and “prevention” refer to the administration of therapy to an individual who may ultimately manifest at least one symptom of a disease, disorder, or condition, but who has not yet done so, to reduce the chance that the individual will develop the symptom of the disease, disorder, or condition over a given period of time. Such a reduction may be reflected, for example, in a delayed onset of the at least one symptom of the disease, disorder, or condition in the patient.

As used herein, recombinant human AC (rhAC) and recombinant human acid ceramidase have a similar meaning, except where the context dictates otherwise.

As used herein, the terms “subject,” “individual” or “patient,” used interchangeably, means any animal, including mammals, such as mice, rats, other rodents, rabbits, dogs, cats, swine, cattle, sheep, horses, or primates, such as humans.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size and sex of the subject. Optimal amounts can also be determined based on monitoring of the subject's response to treatment.

As used herein, the terms “treat,” “treated,” or “treating” mean both therapeutic treatment and prophylactic measures wherein the object is to slow down (lessen) an undesired physiological condition, disorder or disease, or obtain beneficial or desired clinical results. For example, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease.

As used herein, “viral inactivation” comprises a process for inactivation of viruses that may be present in the expression of a recombinant protein from a host cell.

In an embodiment, viral inactivation may be accomplished in a number of ways known in the art. An exemplary process appears below in Tables 1 and 2 below.

TABLE 1
Virus Inactivation Step Description

| | | | 10 L Confirmation |
|---|---|---|---|
| Step | Buffer/Feed | Volume Required | Batch Data |
| Low pH | 1M Citric | 0.0327 mL/mL | 1M Citric Acid: |
| adjustment | Acid | of Harvest | 0.027 mL/mL of Clarified |
| | | | Harvest |
| | | | See Table 8 footnote c |
| Virus | Clarified | | Clarified Harvest Material |
| Inactivation | Harvest | | |
| Sample | | | |
| Neutralization/ | 2M Tris, pH | 0.00775 mL/mL | 2M Tris, pH 9.5: |
| CIEX Load | 9.5 | of Harvest | of 0.044 mL/mL of |
| Adjustment | See Table 8 | | Clarified Harvest to pH |
| | footnote a | | 7.0 |
| CIEX Load | 1M Citric | Not Available | 1M Citric Acid: |
| Adjustment | Acid | | 0.046 mL/mL of |
| | | | Clarified Harvest |

TABLE 2
Virus Inactivation Process Parameters

| Process parameters | | | |
|---|---|---|---|
| Virus inactivation and | | | 10 L |
| CIEX Load | | | Confirmation |
| Adjustment | Target/Set Point | Acceptance Range | Batch Data |
| Temperature (° C.) | Room Temperature | 18-25° C. | 23.8° C. |
| pH virus inactivation | 3.7 | 3.7 ± 0.1 | 3.72 |
| Hold time for virus | 45-60 minutes | 45-60 minutes | 50 minutes |
| inactivation (min) | | | |
| Addition time | Hold completed once Neutralization | | |
| neutralization (min) | buffer introduced | | |
| pH after adjustment | 4.0 ⁽¹⁾ | ±0.1 | 6.99 ⁽¹⁾ |
| Product HOLD duration | 18-25° C. ≤ 24 hrs. | 18-25° C./2-8° C. | 15 hrs. |
| after VI ⁽¹⁾ | or 2-8° C. > 24 hrs. | | |
| | See footnote d | | |
| Product HOLD | 18-25° C. ≤ 24 hrs. | 18-25° C./2-8° C. | 22.4° C. |
| temperature after VI | or 2-8° C. > 24 hrs. | | |
| | See footnote d | | |
| Type of Filter for Post | 0.2 μm PES | | 0.45/0.2 μm |
| VI Fraction | | | PES |
| | | | Filter |
| | | | See footnote b |
| Filter Loading (g/m²) | 500 | ≤1000 | 217 g/m², |
| | | | 268 L/m² |
| | | | See footnote b |

a. The amount of 2 M Tris, pH 9.5 required to get the post low pH VI hold product to pH 7.0 was 4.4% of harvest instead of the estimated 0.775% of harvest material. 1 M Tris Base wild. be used for the 200 L Non-cGMP Protocol to reduce the volume of buffer required for titrations. The estimated volumes are pH 4.0:0.001 L/L of Clarified Filtered Harvest and pH 7.0:0.019 L/L of Clarified Filtered Harvest.

Any method known to the skilled artisan may be used to monitor disease status and the effectiveness the therapy. Clinical monitors of disease status may include but are not limited to ceramide levels, weight, joint length, inflammation, or any other clinical phenotype known to be associated with Farber disease.

b. Post VI Filter used was a Sartopore 2 0.05m2 0.45/0.2 μm PES dual layer filter instead of a single layer 0.2 μm filter. This was changed to add a prefilter to the filter. The original filter loading specification was too optimistic. The Sartopore 2 filter clogged after 13.4 L (10.854 g of Neutralized Post VI low pH hold material). The filter was changed out and replaced with as described herein, any concentration range, percentage range, ratio range or integer range is to be understood to include the value of any integer within the recited range and, when appropriate, fractions thereof (such as one tenth and one hundredth of an integer), unless otherwise indicated. Ranges are approximate and may vary by more than an integer.

c. All estimated titration volumes will be updated in the 200 L Non-cGMP Protocol with the actual L/L volumes required to reflect the actual process used unless the titration buffer is changing.

d. The post viral inactivation product Hold duration and temperature may be changed to 18-25° C. for ≤24 hrs.

Units, prefixes, and symbols are denoted in their Système International de Unites (SI) accepted form. Numeric ranges are inclusive of the numbers defining the range. Measured values are understood to be approximate, taking into account significant digits and the error associated with the measurement.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable subcombination.

### Acid Ceramidase

“AC” refers to the protein encoded by the ASAH1 gene (NCBI UniGene GeneID No. 427). AC hydrolyzes the amide bond linking the sphingosine and fatty acid moieties of the lipid ceramide (Park and Schuchman, 2006, Acid ceramidase and human disease, Biochim. Biophys. Acta., 1758 (12): 2133-2138; Nikolova-Karakashian et al., 2000, Ceramidases, Methods Enzymol. 311:194-201; Hassler et al., 1993, Ceramidases: enzymology and metabolic roles, Adv. Lipid Res. 26:49-57). Mutations of both ASAH1 alleles can lead to Farber's disease.

There are three types of AC described to date (Nikolova-Karakashian et al., 2000). These are classified as acid, neutral, and alkaline ceramidases according to their pH optimum of enzymatic activity. ACs have optimal enzymatic activity at a pH<5. The human AC was the first ceramidase to be cloned (Koch et al., 1996). It is localized in the lysosome and is mainly responsible for the catabolism of ceramide. Dysfunction of this enzyme because of a genetic defect leads to a sphingolipidosis disease called Lipogranulomatosis or Farber disease (Koch et al., 1996; Young et al., 2013, Sphingolipids: regulators of crosstalk between apoptosis and autophagy, J. Lipid. Res. 54:5-19).

AC (N-acylsphingosine deacylase, I.U.B.M.B. Enzyme No. EC 3.5.1.23) protein has been purified from several sources, and the human and mouse cDNAs and genes have been obtained. See Bernardo et al., 1995, Purification, characterization, and biosynthesis of human acid ceramidase, J. Biol. Chem. 270:11098-102; Koch et al., J. Biol. Chem. 1996; Li et al., 1998, Cloning and characterization of the full-length cDNA and genomic sequences encoding murine acid ceramidase, Genomics 50:267-74; Li et al., 1999, The human acid ceramidase gene (ASAH): structure, chromosomal location, mutation analysis and expression, Genomics 62:223-31). It is produced through cleavage of the AC precursor protein (see Ferlinz et al., 2001, Human acid ceramidase: processing, glycosylation, and lysosomal targeting, J. Biol. Chem., 276 (38): 35352-60), which is the product of the ASAH1 gene (NCBI UniGene GeneID No. 427). AC protein [Homo sapiens] (NCBI Accession No. AAC50907) is shown in SEQ ID NO: 1

The AC alpha subunit begins at the amino acid at position 22 and continues through position 142 (as shown in bold in SEQ ID NO: 1 in the Table of Sequences), while the beta subunit of the AC begins with the amino acid at position 143 and continues through position 395 (as shown in italics in SEQ ID NO: 1).

The activity of AC is regulated by cleavage of the inactive precursor polypeptide into the active enzyme consisting of an alpha and beta subunit linked via disulfide bonds (Shtraizent et al., 2008, Autoproteolytic cleavage and activation of human acid ceramidase, J. Biol. Chem. 283:11253-11259). Recombinant AC produced in Chinese Hamster ovary (“CHO”) cells and secreted into the media is a mixture of inactive precursor and active (cleaved) enzyme (He et al., 2003, Purification and characterization of recombinant, human acid ceramidase, J. Biol. Chem. 278:32978-32986). Thus, in the case of AC, the purification process to obtain rhAC may have a large effect on the amount of functional protein obtained based on the relative presence of active and inactive AC. Table 3: provides a listing of certain sequences referenced herein.

TABLE 3
Description of Sequences

| | | |
|---|---|---|
| SEQ | rhAC | MPGRSCVALVLLAAAVSCAV |
| ID | amino acid | AQHAPPWTEDCRKSTYPPSG |
| NO: 1 | sequence. | PTYRGAVPWYTINLDLPPYK |
| | (preprotein) | RWHELMLDKAPVLKVIVNSL |
| | (Bold | KNMINTFVPSGKIMQVVDEK |
| | indicates | LPGLLGNFPGPFEEEMKGIA |
| | signal | AVTDIPLGEIISFNIFYELF |
| | sequence; | TICTSIVAEDKKGHLIHGRN |
| | underline | MDFGVFLGWNINNDTWVITE |
| | indicates | QLKPLTVNLDFQRNNKTVFK |
| | alpha | ASSFAGYVGMLTGFKPGLFS |
| | subunit; | LTLNERFSINGGYLGILEWI |
| | and | LGKKDVMWIGFLTRTVLENS |
| | normal text | TSYEEAKNLLTKTKILAPAY |
| | indicates | FILGGNQSGEGCVITRDRKE |
| | beta | SLDVYELDAKQGRWYVVQTN |
| | subunit) | YDRWKHPFFLDDRRTPAKMC |
| | | LNRTSQENISFETMYDVLST |
| | | KPVLNKLTVYTTLIDVTKGQ |
| | | FETYLRDCPDPCIGW |
| SEQ | rhAC | GGCTCGGTCCGACTATTGCC |
| ID | (DNA) | CGCGGTGGGGGAGGGGGATG |
| NO: 2 | | GATCACGCCACGCGCCAAAG |
| | | GCGATCGCGACTCTCCTTCT |
| | | GCAGGTAGCCTGGAAGGCTC |
| | | TCTCTCTTTCTCTACGCCAC |
| | | CCTTTTCGTGGCACTGAAAA |
| | | GCCCCGTCCTCTCCTCCCAG |
| | | TCCCGCCTCCTCCGAGCGTT |
| | | CCCCCTACTGCCTGGAATGG |
| | | TGCGGTCCCAGGTCGCGGGT |
| | | CACGCGGCGGAGGGGGCGTG |
| | | GCCTGCCCCCGGCCCAGCCG |
| | | GCTCTTCTTTGCCTCTGCTG |
| | | GAGTCCGGGGAGTGGCGTTG |
| | | GCTGCTAGAGCGATGCCGGG |
| | | CCGGAGTTGCGTCGCCTTAG |
| | | TCCTCCTGGCTGCCGCCGTC |
| | | AGCTGTGCCGTCGCGCAGCA |
| | | CGCGCCGCCGTGGACAGAGG |
| | | ACTGCAGAAAATCAACCTAT |
| | | CCTCCTTCAGGACCAACGTA |
| | | CAGAGGTGCAGTTCCATGGT |
| | | ACACCATAAATCTTGACTTA |
| | | CCACCCTACAAAAGATGGCA |
| | | TGAATTGATGCTTGACAAGG |
| | | CACCAGTGCTAAAGGTTATA |
| | | GTGAATTCTCTGAAGAATAT |
| | | GATAAATACATTCGTGCCAA |
| | | GTGGAAAAATTATGCAGGTG |
| | | GTGGATGAAAAATTGCCTGG |
| | | CCTACTTGGCAACTTTCCTG |
| | | GCCCTTTTGAAGAGGAAATG |
| | | AAGGGTATTGCCGCTGTTAC |
| | | TGATATACCTTTAGGAGAGA |
| | | TTATTTCATTCAATATTTTT |
| | | TATGAATTATTTACCATTTG |
| | | TACTTCAATAGTAGCAGAAG |
| | | ACAAAAAAGGTCATCTAATA |
| | | CATGGGAGAAACATGGATTT |
| | | TGGAGTATTTCTTGGGTGGA |
| | | ACATAAATAATGATACCTGG |
| | | GTCATAACTGAGCAACTAAA |
| | | ACCTTTAACAGTGAATTTGG |
| | | ATTTCCAAAGAAACAACAAA |
| | | ACTGTCTTCAAGGCTTCAAG |
| | | CTTTGCTGGCTATGTGGGCA |
| | | TGTTAACAGGATTCAAACCA |
| | | GGACTGTTCAGTCTTACACT |
| | | GAATGAACGTTTCAGTATAA |
| | | ATGGTGGTTATCTGGGTATT |
| | | CTAGAATGGATTCTGGGAAA |
| | | GAAAGATGTCATGTGGATAG |
| | | GGTTCCTCACTAGAACAGTT |
| | | CTGGAAAATAGCACAAGTTA |
| | | TGAAGAAGCCAAGAATTTAT |
| | | TGACCAAGACCAAGATATTG |
| | | GCCCCAGCCTACTTTATCCT |
| | | GGGAGGCAACCAGTCTGGGG |
| | | AAGGTTGTGTGATTACACGA |
| | | GACAGAAAGGAATCATTGGA |
| | | TGTATATGAACTCGATGCTA |
| | | AGCAGGGTAGATGGTATGTG |
| | | GTACAAACAAATTATGACCG |
| | | TTGGAAACATCCCTTCTTCC |
| | | TTGATGATCGCAGAACGCCT |
| | | GCAAAGATGTGTCTGAACCG |
| | | CACCAGCCAAGAGAATATCT |
| | | CATTTGAAACCATGTATGAT |
| | | GTCCTGTCAACAAAACCTGT |
| | | CCTCAACAAGCTGACCGTAT |
| | | ACACAACCTTGATAGATGTT |
| | | ACCAAAGGTCAATTCGAAAC |
| | | TTACCTGCGGGACTGCCCTG |
| | | ACCCTTGTATAGGTTGGTGA |
| | | GCACACGTCTGGCCTACAGA |
| | | ATGCGGCCTCTGAGACATGA |
| | | AGACACCATCTCCATGTGAC |
| | | CGAACACTGCAGCTGTCTGA |
| | | CCTTCCAAAGACTAAGACTC |
| | | GCGGCAGGTTCTCTTTGAGT |
| | | CAATAGCTTGTCTTCGTCCA |
| | | TCTGTTGACAAATGACAGAT |
| | | CTTTTTTTTTTCCCCCTATC |
| | | AGTTGATTTTTCTTATTTAC |
| | | AGATAACTTCTTTAGGGGAA |
| | | GTAAAACAGTCATCTAGAAT |
| | | TCACTGAGTTTTGTTTCACT |
| | | TTGACATTTGGGGATCTGGT |
| | | GGGCAGTCGAACCATGGTGA |
| | | ACTCCACCTCCGTGGAATAA |
| | | ATGGAGATTCAGCGTGGGTG |
| | | TTGAATCCAGCACGTCTGTG |
| | | TGAGTAACGGGACAGTAAAC |
| | | ACTCCACATTCTTCAGTTTT |
| | | TCACTTCTACCTACATATTT |
| | | GTATGTTTTTCTGTATAACA |
| | | GCCTTTTCCTTCTGGTTCTA |
| | | ACTGCTGTTAAAATTAATAT |
| | | ATCATTATCTTTGCTGTTAT |
| | | TGACAGCGATATAATTTTAT |
| | | TACATATGATTAGAGGGATG |
| | | AGACAGACATTCACCTGTAT |
| | | ATTTCTTTTAATGGGCACAA |
| | | AATGGGCCCTTGCCTCTAAA |
| | | TAGCACTTTTTGGGGTTCAA |
| | | GAAGTAATCAGTATGCAAAG |
| | | CAATCTTTTATACAATAATT |
| | | GAAGTGTTCCCTTTTTCATA |
| | | ATTACTCTACTTCCCAGTAA |
| | | CCCTAAGGAAGTTGCTAACT |
| | | TAAAAAACTGCATCCCACGT |
| | | TCTGTTAATTTAGTAAATAA |
| | | ACAAGTCAAAGACTTGTGGA |
| | | AAATAGGAAGTGAACCCATA |
| | | TTTTAAATTCTCATAAGTAG |
| | | CATTCATGTAATAAACAGGT |
| | | TTTTAGTTTGTTCTTCAGAT |
| | | TGATAGGGAGTTTTAAAGAA |
| | | ATTTTAGTAGTTACTAAAAT |
| | | TATGTTACTGTATTTTTCAG |
| | | AAATCAAACTGCTTATGAAA |
| | | AGTACTAATAGAACTTGTTA |
| | | ACCTTTCTAACCTTCACGAT |
| | | TAACTGTGAAATGTACGTCA |
| | | TTTGTGCAAGACCGTTTGTC |
| | | CACTTCATTTTGTATAATCA |
| | | CAGTTGTGTTCCTGACACTC |
| | | AATAAACAGTCACTGGAAAG |
| | | AGTGCCAGTCAGCAGTCATG |
| | | CACGCTGATTGGGTGTGT |
| SEQ | rhAC | AAGCTTACCGCCACCATGAA |
| ID | (DNA) | CTGCTGCATCGGCCTGGGTG |
| NO: 3 | | AGAAGGCGCGTGGCTCGCAC |
| | | CGCGCCAGCTACCCCTCCCT |
| | | GAGCGCCCTCTTCACCGAGG |
| | | CGTCCATCCTCGGATTCGGG |
| | | AGCTTCGCCGTCAAGGCACA |
| | | GTGGACCGAGGATTGCCGCA |
| | | AGAGTACGTACCCCCCCAGT |
| | | GGCCCGACGTACCGCGGCGC |
| | | CGTCCCCTGGTACACGATCA |
| | | ACCTGGACCTCCCCCCGTAC |
| | | AAGCGCTGGCACGAGTTGAT |
| | | GCTGGACAAGGCCCCCGTAC |
| | | TGAAGGTCATCGTGAACTCC |
| | | CTGAAGAACATGATCAACAC |
| | | CTTCGTCCCCTCGGGCAAGA |
| | | TCATGCAGGTCGTGGACGAG |
| | | AAGCTGCCCGGGCTCCTCGG |
| | | CAACTTCCCCGGCCCGTTCG |
| | | AAGAGGAGATGAAGGGCATC |
| | | GCGGCCGTCACTGACATCCC |
| | | CCTGGGCGAGATCATCAGCT |
| | | TCAACATCTTCTACGAGCTG |
| | | TTCACCATCTGCACCTCCAT |
| | | CGTAGCCGAGGACAAGAAGG |
| | | GCCACCTGATCCACGGTCGC |
| | | AACATGGACTTCGGCGTCTT |
| | | CCTGGGCTGGAACATCAACA |
| | | ACGACACCTGGGTCATCACC |
| | | GAGCAGCTGAAGCCGCTCAC |
| | | CGTGAACCTCGATTTCCAGC |
| | | GCAACAACAAGACGGTGTTC |
| | | AAGGCCAGCTCCTTCGCCGG |
| | | GTACGTCGGGATGCTCACGG |
| | | GCTTCAAGCCGGGACTGTTC |
| | | TCGCTGACCCTCAACGAGCG |
| | | GTTCTCCATCAACGGGGGCT |
| | | ACCTCGGCATCCTGGAGTGG |
| | | ATTCTCGGCAAGAAGGACGT |
| | | GATGTGGATCGGCTTCCTCA |
| | | CACGGACCGTGCTGGAAAAC |
| | | TCCACTAGTTACGAGGAGGC |
| | | CAAGAACCTGCTGACCAAGA |
| | | CGAAGATCCTGGCCCCGGCA |
| | | TACTTCATCCTGGGCGGCAA |
| | | CCAGTCGGGCGAGGGGTGCG |
| | | TCATCACCCGCGACCGGAAG |
| | | GAGTCCCTGGACGTCTATGA |
| | | GCTGGACGCCAAGCAGGGCC |
| | | GCTGGTACGTCGTCCAGACG |
| | | AACTACGACCGATGGAAGCA |
| | | CCCCTTCTTCCTCGACGACC |
| | | GGCGCACGCCCGCCAAGATG |
| | | TGCCTGAACCGCACCAGCCA |
| | | GGAGAACATCTCGTTCGAGA |
| | | CGATGTACGACGTGCTGTCG |
| | | ACCAAGCCCGTGCTCAACAA |
| | | GCTGACGGTCTACACCACGC |
| | | TGATCGACGTGACGAAAGGC |
| | | CAGTTCGAAACGTACCTGCG |
| | | GGACTGCCCGGACCCTTGCA |
| | | TCGGCTGGTGATAATCTAGA |
| | | GTCGGGGCGGCCGGCCAAGC |
| | | TTACCGCCACCATGAACTGC |
| | | TGCATCGGGCTGG |
| SEQ | rhAC | GAGAGAAAGCTCGCGGGTCC |
| ID | (DNA) | CACCGGGCCTCCTACCCAAG |
| NO: 4 | | TCTCAGCGCGCTTTTCACCG |
| | | AGGCCTCAATTCTGGGATTT |
| | | GGCAGCTTTGCTGTGAAAGC |
| | | CCAATGGACAGAGGACTGCA |
| | | GAAAATCAACCTATCCTCCT |
| | | TCAGGACCAACGTACAGAGG |
| | | TGCAGTTCCATGGTACACCA |
| | | TAAATCTTGACTTACCACCC |
| | | TACAAAAGATGGCATGAATT |
| | | GATGCTTGACAAGGCACCAG |
| | | TGCTAAAGGTTATAGTGAAT |
| | | TCTCTGAAGAATATGATAAA |
| | | TACATTCGTGCCAAGTGGAA |
| | | AAATTATGCAGGTGGTGGAT |
| | | GAAAAATTGCCTGGCCTACT |
| | | TGGCAACTTTCCTGGCCCTT |
| | | TTGAAGAGGAAATGAAGGGT |
| | | ATTGCCGCTGTTACTGATAT |
| | | ACCTTTAGGAGAGATTATTT |
| | | CATTCAATATTTTTTATGAA |
| | | TTATTTACCATTTGTACTTC |
| | | AATAGTAGCAGAAGACAAAA |
| | | AAGGTCATCTAATACATGGG |
| | | AGAAACATGGATTTTGGAGT |
| | | ATTTCTTGGGTGGAACATAA |
| | | ATAATGATACCTGGGTCATA |
| | | ACTGAGCAACTAAAACCTTT |
| | | AACAGTGAATTTGGATTTCC |
| | | AAAGAAACAACAAAACTGTC |
| | | TTCAAGGCTTCCAGCTTTGC |
| | | TGGCTATGTGGGCATGTTAA |
| | | CAGGATTCAAACCAGGACTG |
| | | TTCAGTCTTACACTGAATGA |
| | | ACGTTTCAGTATAAATGGTG |
| | | GTTATCTGGGTATTCTAGAA |
| | | TGGATTCTGGGAAAGAAAGA |
| | | TGTCATGTGGATAGGGTTCC |
| | | TCACTAGAACAGTTCTGGAA |
| | | AATAGCACAAGTTATGAAGA |
| | | AGCCAAGAATTTATTGACCA |
| | | AGACCAAGATATTGGCCCCA |
| | | GCCTACTTTATCCTGGGAGG |
| | | CAACCAGTCTGGGGAAGGTT |
| | | GTGTGATTACACGAGACAGA |
| | | AAGGAATCATTGGATGTATA |
| | | TGAACTCGATGCTAAGCAGG |
| | | GTAGATGGTATGTGGTACAA |
| | | ACAAATTATGACCGTTGGAA |
| | | ACATCCCTTCTTCCTTGATG |
| | | ATCGCAGAACGCCTGCAAAG |
| | | ATGTGTCTGAACCGCACCAG |
| | | CCAAGAGAATATCTCATTTG |
| | | AAACCATGTATGATGTCCTG |
| | | TCAACAAAACCTGTCCTCAA |
| | | CAAGCTGACCGTATACACAA |
| | | CCTTGATAGATGTTACCAAA |
| | | GGTCAATTCGAAACTTACCT |
| | | GCGGGACTGCCCTGACCCTT |
| | | GTATAGGTTGGTGATAACCT |
| | | AGGGTCGGGGCGGCCGGCC |

In an embodiment, “RVT-801” is a recombinant human acid ceramidase (rhAC) in activated form for the treatment of Farber disease. The alpha and beta subunits of the activated rhAC are joined by a disulfide bond. The molecule is a recombinant human acid ceramidase (rhAC) derived from CHO-M cells transfected with a DNA plasmid vector expressing rhAC. RVT-801 is based on UniProtKB Code: Q13510.

RVT-801 comprises a recombinantly produced acid ceramidase (rhAC) purified to a purity of at least 95% activated form by a process comprising the steps of subjecting the recombinantly produced acid ceramidase to at least two chromatography steps selected from i) cation exchange chromatography; ii) hydrophobic interaction chromatography (HIC); and iii) anion exchange chromatography; and subjecting the recombinantly produced acid ceramidase in solution to one or more viral inactivation steps, wherein the rhAC solution is titrated to a pH of 3.7 or less. The protein sequence of RVT-801 corresponds to SEQ ID NO: 1. In an embodiment the purification of rhAC may be performed in accordance with the processes disclosed in PCT/2018/052463, filed on Sep. 24, 2018, which is incorporated herein by reference in its entirety. The therapeutic effect of RVT-801rhAC has been established in a murine model of severe Farber disease (He, et al, 2017) and has been characterized over multiple studies with endpoints describing positive impacts on histopathological and immunological outcomes along with concomitant reduction of accumulated ceramides.

Other embodiments of active ACs and inactive AC precursor proteins that can be used in this and all aspects of the present invention include, without limitation, those set forth in Table 1 of US 2016/0038574, the contents of which are hereby incorporated by reference.

In some embodiments, the rhAC is a protein that is a protein that is a homolog of SEQ ID NO: 1.

In some embodiments, the rhAC is encoded by a nucleic acid molecule of SEQ ID NO: 2.

In some embodiments, the rhAC is encoded by a nucleic acid molecule of SEQ ID NO: 3.

In some embodiments, the rhAC is encoded by a nucleic acid molecule of SEQ ID NO: 4.

In some embodiments, the sequence of rhAC is as defined in GenBank accession number NM_177924.3 or NM_177924.4, each of which is incorporated by reference in its entirety. The nucleotide sequence encoding the protein can be the complete sequence shown in SEQ ID NO: 2, SEQ ID NO: 3, or SEQ ID NO: 4, or be simply the coding region of the sequence The coding region, for example, could be nucleotides 313 to 1500 of SEQ ID NO: 2 or the corresponding coding region found in SEQ ID NO: 3 or SEQ ID NO: 4. However, as is well known to one of skill in the art, the genetic code is degenerate and, therefore other codons can be used to encode the same protein without being outside of what is disclosed. Since the amino acid sequence is known, any nucleotide sequence that encodes the amino acid sequence is acceptable.

In some embodiments, the nucleotide sequence comprises a signal peptide. In some embodiments, the signal peptide is an amino acid sequence encoded by nucleotides 313 to 375 of SEQ ID NO: 2.

In some embodiments, the protein that is produced comprises a signal peptide of amino acid residues 1-21 of SEQ ID NO: 1.

In some embodiments, the protein that is produced does not comprise a signal peptide, such as the signal peptide of amino acid residues 1-21 of SEQ ID NO: 1. In some embodiments, the signal peptide is removed during a post-translational processing where the enzyme is processed into its different subunits. In some embodiments, the nucleotide sequence is codon optimized for the cell that it the protein is being expressed from. In some embodiments, the protein comprises an alpha-subunit, a beta-subunit, and the like. In some embodiments, the protein that is produced comprises a peptide of amino acid residues 22-142, 45-139, 134-379, 143-395, or 1-395 of SEQ ID NO: 1. The peptide can be a single protein or a polypeptide of different sequences to form the enzyme. In some embodiments, the protein is free of amino acid residues 1-21. These regions can be encoded by a single nucleotide sequence or separate nucleotide sequences or a combination of nucleotide sequences. As discussed herein, any nucleotide sequence encoding the protein can be used and is not limited to the sequence described herein as SEQ ID NO: 2, SEQ ID NO: 3, or SEQ ID NO: 4.

In some embodiments, the rhAC has acid ceramidase (AC) activity but does not have any detectable acid sphingomyelinase activity, such as the rhAC produced in Examples 1 and 2 below. The acid sphingomyelinase activity may be removed, for example, by heat inactivation. See, e.g., U.S. Patent Application Publication No. 20160038574, which is incorporated herein in its entirety. Heat inactivation may also remove other contaminating proteins from an rhAC preparation. Id.

In some embodiments, the purified recombinantly produced acid ceramidase has a purity of at least 90%, 93%, 95%, 98%, or 99%, or a purity of 100%.

In some embodiments, the purified recombinantly produced acid ceramidase has no detectable acid sphingomyelinase activity.

In some embodiments, the acid sphingomyelinase activity of the recombinantly produced acid ceramidase is removed without the use of heat.

Also described herein is a purified recombinantly produced acid ceramidase produced by any of the methods described herein.

Also described herein is a therapeutic composition comprising the purified recombinantly produced acid ceramidase produced by any of the methods described herein.

In some embodiments, the therapeutic composition further comprises a pharmaceutically acceptable carrier.

In some embodiments, the therapeutic composition further comprises one or more pharmaceutically acceptable adjuvants, excipients, or stabilizers. In some embodiments, the one or more pharmaceutically acceptable adjuvants, excipients, or stabilizers comprise one or more of trisodium citrate, citric acid, human serum albumin, mannitol, sodium phosphate monobasic, sodium phosphate dibasic, polysorbate, sodium chloride, histidine, sucrose, trehalose, glycine, and water. In some embodiments, the salts are hydrates (e.g., trisodium citrate dihydrate, citric acid monohydrate, sodium phosphate monobasic monohydrate, and/or sodium phosphate dibasic heptahydrate).

In some embodiments, the therapeutic composition further comprises one or more additional agents that reduce ceramide levels and/or reduce inflammation associated with Farber disease or that reduces pro-inflammatory neutrophils and/or activated monocytes.

Also described herein is a method of treatment of a disease or disorder associated with reduced or absent acid ceramidase and increased pro-inflammatory neutrophils and/or activated monocytes, or with inflammation associated with Farber disease, comprising administering an effective amount of any of the therapeutic compositions described herein in vivo to a subject in need thereof or in vitro to a population of cells. In some embodiments, the therapeutic composition is administered in vivo to a subject in need thereof. In some embodiments, the therapeutic composition is administered in vitro to a population of cells.

The term “homolog” refers to protein sequences having between 80% and 100% sequence identity to a reference sequence. Percent identity between two peptide chains can be determined by pair wise alignment using the default settings of the AlignX module of Vector NTI v.9.0.0 (Invitrogen Corp., Carslbad, Calif.). In some embodiments, the homolog has at least, or about, 80, 85, 86, 87, 88, 89, 90, 91, 92, 93, 94, 95, 96, 97, 98, or 99% identity to a sequence described herein, such as SEQ ID NO: 1. In some embodiments, the protein delivered to the subject conservative substitutions as compared to a sequence described herein. Non-limiting exemplary conservative substitutions are shown in Table 4 are encompassed within the scope of the disclosed subject matter. Substitutions may also be made to improve function of the enzyme, for example stability or enzyme activity. Conservative substitutions will produce molecules having functional and chemical characteristics similar to those molecules into which such modifications are made. Exemplary amino acid substitutions are shown in Table 4 below.

TABLE 4
Exemplary Conservative Substitutions:

| Original Residue | Exemplary Conservative Substitutions |
|---|---|
| Ala | Val, Leu, Ile |
| Arg | Lys, Gln, Asn |
| Asn | Gln |
| Asp | Glu |
| Cys | Ser, Ala |
| Gln | Asn |
| Gly | Pro, Ala |
| His | Asn, Gln, Lys, Arg |
| Ile | Leu, Val, Met, Ala, Phe |
| Leu | Ile, Val, Met, Ala, Phe |
| Lys | Arg, Gln, Asn |
| Met | Leu, Phe, Ile |
| Phe | Leu, Val, Ile, Ala, Tyr |
| Pro | Ala |
| Ser | Thr, Ala, Cys |
| Thr | Ser |
| Trp | Tyr, Phe |
| Tyr | Trp, Phe, Thr, Ser |
| Val | Ile, Met, Leu, Phe, Ala |

As used herein, “active acid ceramidase” “activated rhAC,” “rhAC in activated form” or “active AC” refers to AC precursor proteins that has undergone autoproteolytic cleavage into the active form (composed of α- and β-subunits). The mechanism of human AC cleavage and activation is reported in Shtraizent et al., 2008. Activation is promoted by the intracellular environment, and, based on highly conserved sequences at the cleavage site of ceramidase precursor proteins across species, is expected to occur in most, if not all, cell types. Activation may also be achieved through downstream purification processing conditions, such as viral inactivation processes at an acidic pH.

As used herein, “inactive acid ceramidase,” “inactive AC,” or “inactive acid ceramidase precursor,” “inactive AC precursor,” or (AC preprotein) refers to AC precursor protein that has not undergone autoproteolytic cleavage into the active form.

Inactive AC precursors and active ACs suitable for use in the recombinant acid ceramidase of this and all aspects of the present invention can be homologous (i.e., derived from the same species) or heterologous (i.e., derived from a different species) to the tissue, cells, and/or subject being treated. Acid ceramidase (e.g., AC) precursor proteins undergo autoproteolytic cleavage into the active form (composed of α- and β-subunits). The mechanism of human AC cleavage and activation is reported in Shtraizent et al., 2008. Thus, ceramidase as used herein includes both active ceramidase and ceramidase precursor proteins, where ceramidase precursor proteins are converted into active ceramidase proteins through autoproteolytic cleavage. Embodiments in which the precursor protein is taken up by the cell of interest and converted into active ceramidase thereby, as well as embodiments in which the precursor protein is converted into active ceramidase by a different cell or agent (present, for example, in a culture medium), are both contemplated.

Active ACs and inactive AC precursor proteins that can be used in this and all aspects of the present invention include, without limitation, those set forth in Table 1 of Schuchman, E. H. (inventor), Icahn School of Medicine at Mount Sinai (applicant), 2016, Feb. 11, Therapeutic Acid Ceramidase Compositions And Methods Of Making And Using Them, published as U.S. Published Patent Application No. US 2016/0038574 A1.

The purified recombinant acid ceramidase of the therapeutic composition may, in some instances, contain a lesser amount of inactive AC precursor than active AC.

In some embodiments, the amount of the active rhAC may be greater than 95 wt. %, or greater than 96 wt. %, or greater than 97 wt. %, or greater than 98 wt. %, or greater than 99 wt. %, or greater than 99.5 wt. % or substantially 100 wt. %.

As used herein, “recombinant human acid ceramidase” or “rhAC” refers to protein encoded by the human ASAH1 gene and produced by the process described herein. The amino acid sequence of rhAC (AC preprotein) is SEQ ID NO: 1.

As used herein, “acid sphingomyelinase activity” or “ASM activity” refers to a related lipid hydrolase that tightly binds to AC and co-purifies with it (Bernardo et al., 1995).

Treatment according to this aspect of the present invention is carried out using methods that will be apparent to the skilled artisan. For a discussion of AC in the context of human disease, see Park and Schuchman, 2006; and Zeidan et al., 2008, Molecular targeting of acid ceramidase: implications to cancer therapy, Curr. Drug Targets, 9 (8): 653-661).

The treatment with the human recombinant AC produced by the methods of the present invention may be accompanied with a pre-treatment, or treatment with an antipyretic, antihistamine and/or corticosteroid to reduce occurrence of adverse effects.

In some embodiments, treatment is carried out by introducing a ceramidase protein into the cells. An approach for delivery of proteins or polypeptide agents (e.g., active ceramidase, inactive ceramidase precursor proteins) involves the conjugation of the desired protein or polypeptide to a polymer that is stabilized to avoid enzymatic degradation of the conjugated protein or polypeptide. Conjugated proteins or polypeptides of this type are described in Ekwuribe, N. N. (inventor), Protein Delivery, Inc. (assignee), 1997 Oct. 28, Conjugation-stabilized therapeutic agent compositions, delivery and diagnostic formulations comprising same, and method of making and using the same, published as U.S. Pat. No. 5,681,811.

### Dosage and Administration

For example, site specific administration may be to body compartment or cavity such as intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, vaginal, rectal, buccal, sublingual, intranasal, or transdermal means.

The recombinant AC purified by the methods of the invention may be formulated into a powder or cake to be dissolved and administered as an injection or an infusion or may be formulated directly as a liquid composition. The composition may also be formulated into an inhaled formulation.

Embodiments disclosed herein provide methods for treating inflammation associated with Farber disease in a subject in need thereof, the methods comprising administering to the subject a pharmaceutical composition comprising a recombinant human acid ceramidase (rhAC) in an effective amount of about 3, 10, or 50 mg/kg in, for example, once a week, once every two weeks, or once a month repeat dosages for the duration of subject's life.

In certain aspects of the invention, the treatment is started when the subject is under 1, 2, 3, 4, 5, 6, 7, 8, 9, or 10 years of age, or between 1 and 2, 3, 4, 5, 6, 7, 8, 9, 10, 25, 50, 60, 70 or 80 years of age (e.g., between 1 and 2, between 1 and 3, etc.). In some embodiments, the subject is between 16 and 61. In some embodiments, the subject starts treatment at age 16. In some embodiments, the subject is between 12 and 69. In some embodiments, the subject starts treatment at age 12. In some embodiments, the subject is between 19 and 74. In some embodiments, the subject starts treatment at age 19. In some embodiments, the subject is between 4 and 62. In some embodiments, the subject starts treatment at age 4. In some embodiments, the subject is between 7 and 42. In some embodiments, the subject starts treatment at age 7. In some embodiments, the subject is between 1 and 6 months. In some embodiments, the subject starts treatment at age 1 month, 2 months, 3 months, 4 months, 5 months, or 6 months. In some embodiments, the subject is between 6 and 43. In some embodiments, the subject starts treatment at age 6. In some embodiments, the subject is between 5 and 31. In some embodiments, the subject starts treatment at age 5. In some embodiments, the subject is between 5 and 57. In some embodiments, the subject is between 5 and 29. In some embodiments, the subject is between 1 and 3. In some embodiments, the subject starts treatment at age 1. In some embodiments, the subject is between 10 and 70. In some embodiments, the subject starts treatment at age 10. In some embodiments, the subject is between 5 and 80, between 10 and 70, between 20 and 75, between 5 and 60, or between 5 and 30 years of age.

In general, if administering a systemic dose of the protein, it is desirable to provide the recipient with a dosage of protein which is in the range of from about 1 ng/kg-100 ng/kg, 100 ng/kg-500 ng/kg, 500 ng/kg-1 μkg, 1 pkg/kg-100 μkg/kg, 100 pkg/kg-500 μkg/kg, 500 μkg/kg-1 mg/kg, 1 mg/kg-50 mg/kg, 50 mg/kg-100 mg/kg, 100 mg/kg-500 mg/kg (body weight of recipient), although a lower or higher dosage may be administered.

In some embodiments, the effective amount of rhAC that is administered is about 0.1 mg/kg to about 10 mg/kg. In some embodiments, the effective amount is about 1 mg/kg to about 5 mg/kg. In some embodiments, the effective amount is about 10 mg/kg to about 50 mg/kg. In some embodiments, the effective amount is about 10 mg/kg to about 20 mg/kg. In some embodiments, the effective amount is about 20 mg/kg to about 30 mg/kg. In some embodiments, the effective amount is about 30 mg/kg to about 40 mg/kg. In some embodiments, the effective amount is about 40 mg/kg to about 50 mg/kg. In some embodiments, the effective amount is about 1, 2, 3, 4, 5, 6, 7, 8, 9, or 10 mg/kg.

In some embodiments, a subject diagnosed with Farber disease is administered rhAC at about 1 mg/kg to about 5 mg/kg rhAC or about 2 mg/kg to about 5 mg/kg rhAC every two weeks. In one embodiment, the dosage escalates from 1 mg/kg or 2 mg/kg to 5 mg/kg at week 4. If a dose level is not tolerated by an individual subject, the dose for that subject may be reduced from 2 mg/kg to 1 mg/kg, or 5 mg/kg to 2 mg/kg, as appropriate. The rhAC may be administered every 2 weeks for at least 10, 20, or 30 weeks or for the duration of the subject's life. In one exemplary embodiment, a subject is diagnosed with Farber disease and is identified as having: 1) subcutaneous nodules; and/or 2) an acid ceramidase activity value in white blood cells, cultured skin fibroblasts or other biological sources (e.g., plasma) that is less than 30% of control values; and/or 3) nucleotide changes within both alleles of the acid ceramidase gene (ASAH1) that indicate, through bioinformatic, gene expression studies, and/or other methods, a possible loss of function of the acid ceramidase protein. In some embodiments, the subject is administered rhAC every two weeks for 28 weeks. In some embodiments, the delivery of rhAC is by intravenous infusion (e.g., saline infusion). In some embodiments, starting at about 2 mg/kg and escalating to about 5 mg/kg rhAC (e.g., to 5 mg/kg at week 4).

In additional embodiments, method for treating inflammation associated with Farber disease in a subject in need thereof are disclosed, the methods comprising administering to the subject a pharmaceutical composition comprising a recombinant human acid ceramidase (rhAC) in an effective amount of about 1 mg to about 5 mg/kg or about 2 mg/kg to about 5 mg/kg in, for example, once a week, once every two weeks, or once a month repeat dosages for at least 10 or at least 20 weeks, for 28 weeks, or for the duration of subject's life. In some embodiments, the administration is by intravenous infusion. In one embodiment, the method of treating Farber disease in a subject in need thereof comprises administering to the subject a pharmaceutical composition comprising a recombinant human acid ceramidase (rhAC) in an effective amount of about 1 mg to about 5 mg/kg or about 2 mg/kg to about 5 mg/kg in, for example, once a week, once every two weeks, or once a month repeat dosages for at least 10 or 20 weeks, for 28 weeks, or for the duration of subject's life.

In certain aspects of the invention, the treatment is started when subject is under one year of age.

In aspects of the invention, the treatment is started when the subject is between 1 and 5 years of age.

In some embodiments, efficacy of treatment is assessed by any of the following means:

Percent change from baseline in net nodule (≥5 mm) count after treatment with rhAC for 28 weeks:

Percent change from baseline in net nodule (≥10 mm) count and comparison to placebo after treatment with rhAC for 28 weeks:

Percent change from baseline in total nodule count (regardless of size) and comparison to placebo after treatment with rhAC for 28 weeks:

Change and percent change from baseline of joint range of motion in selected joints and comparison to placebo after treatment with rhAC for 28 weeks:

Change and percent change from baseline of 6 minute walk distance and comparison to placebo after treatment with rhAC for 28 weeks:

Change and percent change from baseline of pulmonary function tests and comparison to placebo after treatment with rhAC for 28 weeks:

Change and percent change from baseline of FDT score and comparison to placebo after treatment with rhAC for 28 weeks:

Change and percent change from baseline in Z score of body weight and height for age during treatment with rhAC or placebo over 28 weeks.

### Pharmaceutical Compositions

The therapeutic composition may also include pharmaceutically acceptable adjuvants, excipients, and/or stabilizers, and can be in solid or liquid form, such as tablets, capsules, powders, solutions, suspensions, or emulsions. Such additional pharmaceutically acceptable ingredients have been used in a variety of enzyme replacement therapy compositions and include, without limitation, trisodium citrate, citric acid, human serum albumin, mannitol, sodium phosphate monobasic, sodium phosphate dibasic, polysorbate, sodium chloride, histidine, sucrose, trehalose, glycine, and/or water for injections. In some embodiments, the salts are hydrates (e.g., trisodium citrate dihydrate, citric acid monohydrate, sodium phosphate monobasic monohydrate, and/or sodium phosphate dibasic heptahydrate).

In some embodiments, the pharmaceutical composition is administered as described herein. For example, in some embodiments, the composition is administered to a subject orally, by inhalation, by intranasal instillation, topically, transdermally, parenterally, subcutaneously, intravenous injection, intra-arterial injection, intramuscular injection, intraplurally, intraperitoneally, intrathecally, or by application to a mucous membrane.

The therapeutic compositions described herein can be prepared for use for parenteral (subcutaneous, intramuscular or intravenous) or any other administration particularly in the form of liquid solutions or suspensions. The formulation can also be suitable for an injectable formulation. In some embodiments, the injectable formulation is sterile. In some embodiments, the injectable formulation is pyrogen free. In some embodiments, the formulation is free of other antibodies that bind to other antigens other than an antigen described herein.

Suitable vehicles and their formulation and packaging are described, for example, in Remington: The Science and Practice of Pharmacy (21st ed., Troy, D. ed., Lippincott Williams & Wilkins, Baltimore, Md. (2005) Chapters 40 and 41). Additional pharmaceutical methods may be employed to control the duration of action. Controlled release preparations may be achieved through the use of polymers to complex or absorb the compounds.

Another possible method to control the duration of action by controlled release preparations is to incorporate the compounds of into particles of a polymeric material such as polyesters, polyamino acids, hydrogels, poly(lactic acid) or ethylene vinylacetate copolymers. Alternatively, instead of incorporating these agents into polymeric particles, it is possible to entrap these materials in microcapsules prepared, for example, interfacial polymerization, for example, hydroxymethylcellulose or gelatin-microcapsules and poly(methylmethacylate)-microcapsules, respectively, or in colloidal drug delivery systems, for example, liposomes, albumin microspheres, microemulsions, nanoparticles, and nanocapsules or in macroemulsions.

The purified recombinant AC may be orally administered, for example, with an inert diluent, or with an assimilable edible carrier, or they may be enclosed in hard or soft shell capsules, or they may be compressed into tablets, or may be incorporated directly with the food of the diet. For oral therapeutic administration, the purified recombinant AC may be incorporated with excipients and used in the form of tablets, capsules, elixirs, suspensions, syrups, and the like. Such compositions and preparations should contain at least 0.1% of ceramidase. The percentage of purified recombinant AC in these compositions may, of course, be varied and may conveniently be between 2% to 60% of the weight of the unit. The amount of the purified recombinant AC in such therapeutically useful compositions is such that a suitable dosage will be obtained.

The tablets, capsules, and the like may also contain a binder such as gum tragacanth, acacia, corn starch, or gelatin: excipients such as dicalcium phosphate: a disintegrating agent such as corn starch, potato starch, or alginic acid: a lubricant such as magnesium stearate; and a sweetening agent such as sucrose, lactose, or saccharin. When the dosage unit form is a capsule, it may contain, in addition to materials of the above type, a liquid carrier, such as fatty oil.

The purified recombinant AC may also be administered parenterally. Solutions or suspensions of ceramidase can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. Liquid carriers include, but are not limited to, water, saline, aqueous dextrose and related sugar solutions, and glycols such as propylene glycol or polyethylene glycol, for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

Kits, which are described herein and below, are also provided which are useful for carrying out embodiments described herein. In some embodiments, the kits comprise a first container containing or packaged in association with the above-described polypeptides. The kit may also comprise another container containing or packaged in association solutions necessary or convenient for carrying out the embodiments. The containers can be made of glass, plastic or foil and can be a vial, bottle, pouch, tube, bag, etc. The kit may also contain written information, such as procedures for carrying out the embodiments or analytical information, such as the amount of reagent contained in the first container means. The container may be in another container apparatus, e.g. a box or a bag, along with the written information.

Yet another aspect provided for herein is a kit for method for treating inflammation associated with Farber disease in a subject in need thereof. In some embodiments, the kit comprises at least one container comprising a rhAC polypeptide or a nucleic acid molecule encoding the same. In some embodiments, the kit comprises a container comprising a cell that is configured to express rhAC. In some embodiments, the cell is a CHO cell. In some embodiments, the kit comprises conditioned media from a cell that expresses rhAC. In some embodiments, the conditioned media is from a CHO cell.

The purified recombinant AC may also be administered directly to the airways in the form of an aerosol. For use as aerosols, ceramidase in solution or suspension may be packaged in a pressurized aerosol container together with suitable propellants, for example, hydrocarbon propellants like propane, butane, or isobutane with conventional adjuvants. The purified recombinant AC may also be administered in a non-pressurized form.

Exemplary delivery devices include, without limitation, nebulizers, atomizers, liposomes (including both active and passive drug delivery techniques) (Wang et al., 1997, pH-sensitive immunoliposomes mediate target-cell-specific delivery and controlled expression of a foreign gene in mouse, Proc. Nat'l Acad. Sci. USA 84:7851-5); Bangham et al., 1965, Diffusion of univalent ions across the lamellae of swollen phospholipids, J. Mol. Biol. 13:238-52; Hsu C. C. (inventor), Genentech, Inc. (assignee), 1997 Aug. 5. Method for preparing liposomes, published as U.S. Pat. No. 5,653,996; Lee, K.-D., et al. (inventors), President and Fellows of Harvard College and the University of Pennsylvania, Intracellular delivery of macromolecules, published as U.S. Pat. No. 5,643,599: Holland J. W. (inventor), The University of British Columbia, Bilayer stabilizing components and their use in forming programmable fusogenic liposomes, published as U.S. Pat. No. 5,885,613: Dzau, V. J, and Kaneda, Yasufumi (inventors), Method for producing in vivo delivery of therapeutic agents via liposomes, published as U.S. Pat. No. 5,631,237; and Loughrey, et al. (inventors), The Liposome Company (assignee), Preparation of targeted liposome systems of a defined size distribution, published as U.S. Pat. No. 5,059,421: Wolff et al., 1984, The use of monoclonal anti-Thy 1 IgG1 for the targeting of liposomes to AKR-A cells in vitro and in vivo, Biochim. Biophys. Acta 802:259-73), transdermal patches, implants, implantable or injectable protein depot compositions, and syringes. Other delivery systems which are known to those of skill in the art can also be employed to achieve the desired delivery of ceramidase to the desired organ, tissue, or cells.

Administration can be carried out as frequently as required and for a duration that is suitable to provide effective treatment. For example, administration can be carried out with a single sustained-release dosage formulation or with multiple daily doses.

Treatment according to this and all aspects of the present invention may be carried out in vitro or in vivo. In vivo treatments include, for example, embodiments in which the population of cells is present in a mammalian subject. In such embodiments, the population of cells can be either autologous (produced by the subject), homologous, or heterologous. Suitable subjects according to these embodiments include mammals, e.g., human subjects, equine subjects, porcine subjects, feline subjects, and canine subjects.

The effective amount of a therapeutic agent/cell population of the present invention administered to the subject will depend on the type and severity of the disease or disorder and on the characteristics of the individual, such as general health, age, sex, body weight, and tolerance to drugs. It will also depend on the degree, severity, and type of disease or disorder. The skilled artisan will be able to determine appropriate dosages depending on these and other factors.

The purified recombinant AC in all aspects of the invention can be produced using ACs set forth in Table 1 of Schuchman, US 2016/0038574 A1, as noted above. In this and all aspects of the present invention (including the in vivo methods discussed below), the AC can be homologous (i.e., derived from the same species) or heterologous (i.e., derived from a different species) to the one or more cells being treated. The human AC is preferred in methods for human therapy.

The rhAC of the present invention may be produced in accordance with the processes of Schuchman, E. H. (inventor), Icahn School of Medicine at Mount Sinai (applicant), Recombinant Human Acid Ceramidase Production Process. International application under the Patent Cooperation Treaty, PCT/US2018/052463 (not yet published), the contents of which is hereby incorporated by reference in its entirety.

In some aspects of the methods of the invention, the human recombinant AC is produced in CHO cells using the methods described in LeFourm V., et al. (inventors), Selexis S. A. (applicant), 2014 Aug. 2, Enhanced Transgene Expression And Processing, International application under the Patent Cooperation Treaty, published as WO2014/118619, incorporated herein by reference in its entirety.

Generally, the use of recombinant expression systems involves inserting the nucleic acid molecule encoding the amino acid sequence of the desired peptide into an expression system to which the molecule is heterologous (i.e., not normally present). One or more desired nucleic acid molecules encoding a peptide of the invention may be inserted into the vector. When multiple nucleic acid molecules are inserted, the multiple nucleic acid molecules may encode the same or different peptides. The heterologous nucleic acid molecule is inserted into the expression system or vector in proper sense (5′→3′) orientation relative to the promoter and any other 5′ regulatory molecules, and correct reading frame.

The preparation of the nucleic acid constructs can be carried out using standard cloning procedures well known in the art as described by Sambrook, J., et al., Molecular Cloning: A laboratory manual (Cold Springs Harbor 1989). Cohen and Boyer (inventors), Board of Trustees of the Leland Stanford Jr. University (assignee), Process for producing biologically functional molecular chimeras, published as U.S. Pat. No. 4,237,224, describes the production of expression systems in the form of recombinant plasmids using restriction enzyme cleavage and ligation with DNA ligase. These recombinant plasmids are then introduced by means of transformation into a suitable host cell.

A variety of genetic signals and processing events that control many levels of gene expression (e.g., DNA transcription and messenger RNA (mRNA) translation) can be incorporated into the nucleic acid construct to maximize peptide production. For the purposes of expressing a cloned nucleic acid sequence encoding a desired recombinant protein, it is advantageous to use strong promoters to obtain a high level of transcription. Depending upon the host system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the PR and PL promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene. Common promoters suitable for directing expression in mammalian cells include, without limitation, SV40, MMTV, metallothionein-1, adenovirus Ela, CMV, immediate early, immunoglobulin heavy chain promoter and enhancer, and RSV-LTR. Mammalian cells that may be used for manufacture of the recombinant protein of the present invention include, for example, Chinese Hamster Ovary (CHO) cells, plant cells, chicken eggs, and human fibroblasts.

There are other specific initiation signals required for efficient gene transcription and translation in prokaryotic cells that can be included in the nucleic acid construct to maximize peptide production. Depending on the vector system and host utilized, any number of suitable transcription and/or translation elements, including constitutive, inducible, and repressible promoters, as well as minimal 5′ promoter elements, enhancers or leader sequences may be used. For a review on maximizing gene expression see Roberts and Lauer, 1979, Maximizing gene expression on a plasmid using recombination in vitro, Methods in Enzymology 68:473-82.

A nucleic acid molecule encoding a recombinant protein of the present invention, a promoter molecule of choice, including, without limitation, enhancers, and leader sequences: a suitable 3′ regulatory region to allow transcription in the host, and any additional desired components, such as reporter or marker genes, are cloned into the vector of choice using standard cloning procedures in the art, such as described in Sambrook, J., et al., 1989; Ausubel, F. M., 1999, Short Protocols in Molecular Biology (Wiley 1999), and Cohen and Boyer, U.S. Pat. No. 4,237,224.

Once the nucleic acid molecule encoding the peptide has been cloned into an expression vector, it is ready to be incorporated into a host. Recombinant molecules can be introduced into cells, without limitation, via transfection (if the host is a eukaryote), transduction, conjugation, mobilization, or electroporation, lipofection, protoplast fusion, mobilization, or particle bombardment, using standard cloning procedures known in the art, such as described by Sambrook, J., et al., 1989.

A variety of suitable host-vector systems may be utilized to express the recombinant protein or polypeptide. Primarily, the vector system must be compatible with the host used. Host-vector systems include, without limitation, the following: bacteria transformed with bacteriophage DNA, plasmid DNA, or cosmid DNA: microorganisms such as yeast containing yeast vectors: mammalian cell systems infected with virus (e.g., vaccinia virus, adenovirus, etc.); insect cell systems infected with virus (e.g., baculovirus); and plant cells infected by bacteria.

Purified peptides may be obtained by several methods readily known in the art, including ion exchange chromatography, hydrophobic interaction chromatography, affinity chromatography, gel filtration, and reverse phase chromatography. In some embodiments, the peptide is produced in purified form (for example, at least 80%, 85%, 90% or 95% pure) by conventional techniques. Depending on whether the recombinant host cell is made to secrete the peptide into growth medium (see Bauer, et al. (inventor), Cornel Research Foundation, Inc. (assignee), 2003 Jul. 22, published as U.S. Pat. No. 6,596,509), the peptide can be isolated and purified by centrifugation (to separate cellular components from supernatant containing the secreted peptide) followed by sequential ammonium sulfate precipitation of the supernatant. In one embodiment of the present invention, cells may be transformed with DNA encoding AC and then cultured under conditions effective to produce the medium containing inactive AC precursor. The fraction containing the peptide is subjected to gel filtration in an appropriately sized dextran or polyacrylamide column to separate the peptides from other proteins. If necessary, the peptide fraction may be further purified by other chromatography.

The foregoing embodiments and advantages are merely exemplary and are not to be construed as limiting the present invention. The present teachings can be readily applied to other types of apparatuses, experiments and surgical procedures. Also, the description of the embodiments of the present invention is intended to be illustrative and not to limit the scope of the claims. Many alternatives, modifications, and variations will be apparent to those skilled in the art.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### EXAMPLES

Example 1. Wild type, Farber mouse, and Farber mouse treated with a recombinant human acid ceramidase (RVT-801) were analyzed for immune cell population makeup. The Farber mouse model was used, as it is a “knock-in” mouse model established on a W4/129Sv/CD-1 background with a single nucleotide missense mutation identified in a severe-onset FD patient to create a homozygous Asah1P36IR P36IR animal that produced a non-functional version of acid ceramidase. This disease model recapitulates monocytic infiltration of multiple tissues and is therefore useful to study the immune environment of Farber disease using this diseases model.

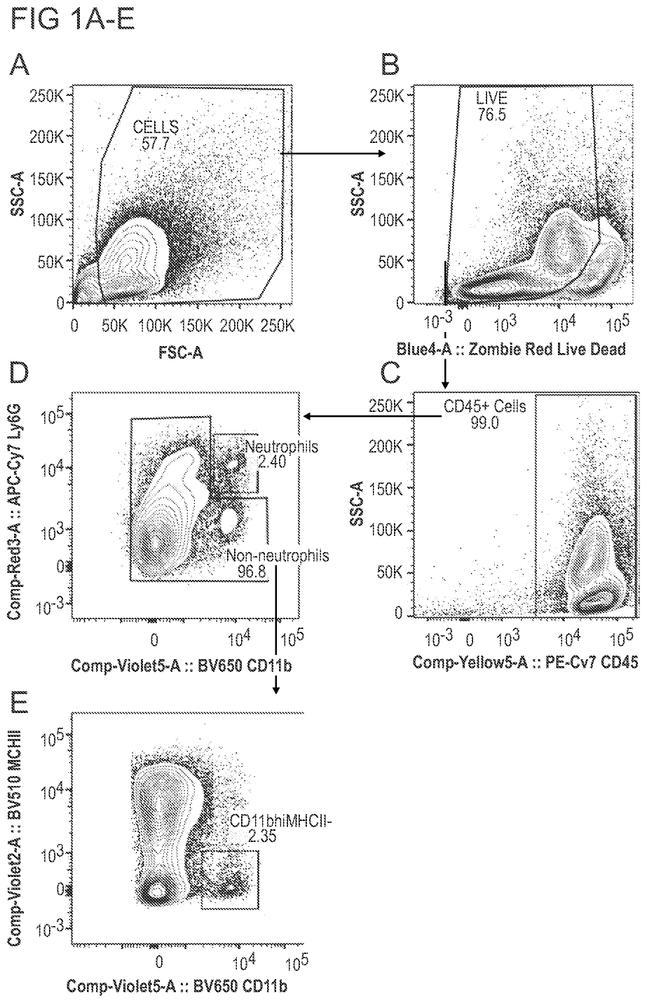

Farber mice (genotype confirmed by PCR) were dosed with 4-once weekly intraperitoneal (IP) doses of 10 mg/kg/dose recombinant human acid ceramidase (RVT-801) beginning just after weaning (aged 3-4 weeks) and were sacrificed for necropsy following their 4th and final RVT-801 administration (at 7 weeks of age). Control wild type and Farber mice were not dosed with vehicle and three control animals of each genotype were necropsied and samples collected for assessment at 4 or 8 weeks of age. At the indicated time points, Farber mice and littermate controls (WT) were harvested to assess the composition of immune cells in key tissues of ceramide accumulation (spleen, liver and lung). In addition, blood was collected to correlate the tissue-specific inflammation in the periphery. Samples were processed to a single cell suspension, stained according to Table 5 (Panel 1) and Table 6 (Panel 2) described below, and run on an BD LSRII flow cytometer. Single stained samples (compensation beads) and fluorescence minus one (FMO) samples served as controls for the study. Raw data files were analyzed using FlowJo v10 and a representative gating strategy is shown as FIG. 1.

Results. FIG. 1 cell populations that were first gated based on size (SSC×FSC) to remove cellular debris from processing. This population was further gated based on live and dead cells to remove the cell population that was positive for the Zombie red dye. The live cells were then gated to select the CD45+ population. This population was further gated to determine the percent of CD45⁺ cells that were Ly6G and CD11b double positive; or neutrophils. The remaining population was selected and gated to select for the CD11b⁺MHCII⁻ population to determine the population of activated monocytes per sample type. This was done for whole blood, spleen, liver, and lung samples in this way using FlowJo v10. Lung samples were further gated to select for activated macrophages.

TABLE 5
Neutrophil Panel

| Marker | Clone | Fluorophore |
|---|---|---|
| CCR2 | SA203G11 | BV421 |
| CD68 | FA-11 | Alexa 647 |
| CD11c | N418 | BV711 |
| MHCII | M5/114.15.2 | BV510 |
| CD86 | GL-1 | BV605 |
| CD206 | C068C2 | Alexa488 |
| CD11b | M1/70 | BV650 |
| CD45 | 104 | PE-Cy7 |
| Ly6G | 1A8 | APC-Cy7 |
| Ly6C | HK1.4 | PE-CF594 |
| CX3CR1 | SA011F11 | PE |
| F4/80 | BM8 | PE-Cy5 |
| CD23 | B3B4 | Alexa700 |

TABLE 6
Monocyte Panel

| Marker | Clone | Fluorophore |
|---|---|---|
| CD3 | 500A2 | Alexa700 |
| CD19 | 6D5 | Alexa488 |
| CD4 | GK1.5 | APC-Cy7 |
| CD8 | 43-6.7 | BV650 |
| CD62L | MEL-14 | BV605 |
| CD44 | IM7 | PE |
| CD45 | 104 | PE-Cy7 |
| CCR7 | 4B12 | PE-Cy5 |
| CD127 | A7R34 | BV711 |
| MHCII | M5/114.15.2 | BV510 |
| CD38 | 90 | Alexa647 |
| CCR6 | BV421 | 29-2L17 |
| CXCR3 | CXCR3-173 | PE-CF594 |

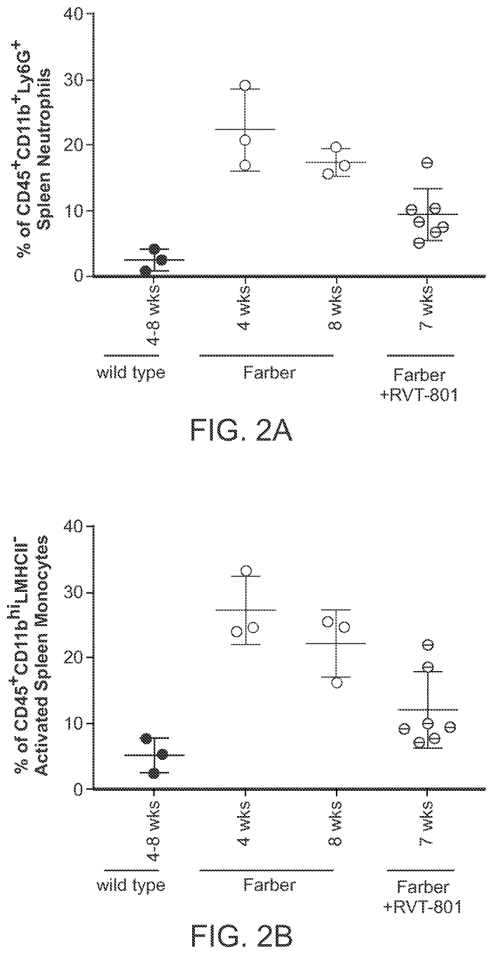

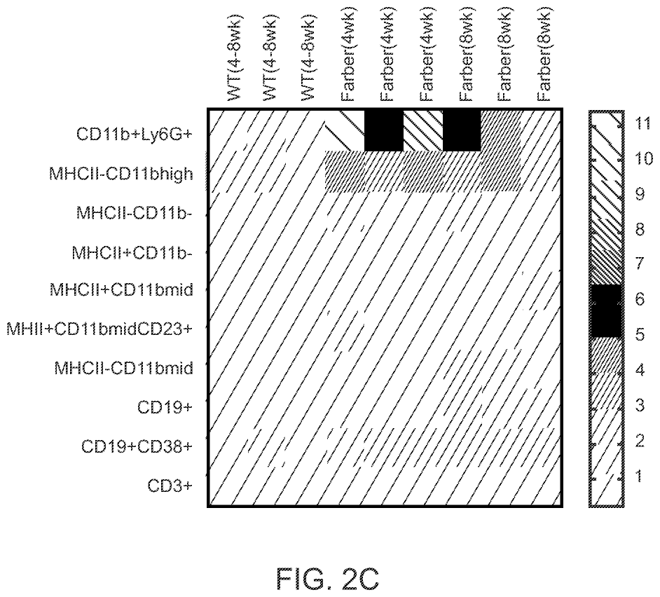

Example 2. Splenic immune cell populations. Results are depicted in FIG. 2. Inflammatory cell populations which are characteristic of an inflammatory state were analyzed from control 4 and 8 week old wild-type and Farber mouse spleens. The population of Ly6GCD11b double positive CD45⁺ neutrophils and CD11b⁺MHCII-CD45⁺ activated monocytes were determined from 7 week old Farber mice that were administered 10 mg/kg/dose RVT-801 once weekly beginning at 3 weeks of age for a total of 4 doses over 4 weeks. Heat map analysis of fold change differences in the frequency of immune cells in the spleen of age-matched Farber mice and littermate controls (WT). Each column represents an individual animal. Fold change was calculated by setting the average WT value to 1.

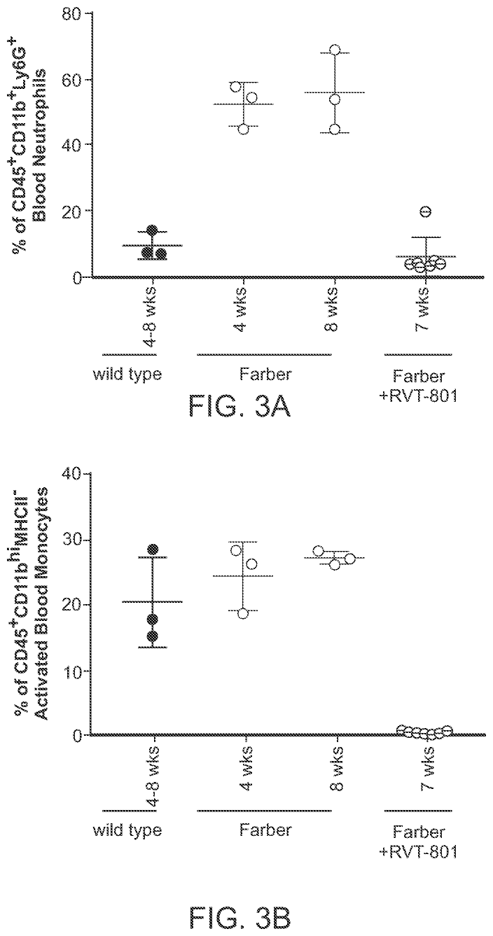

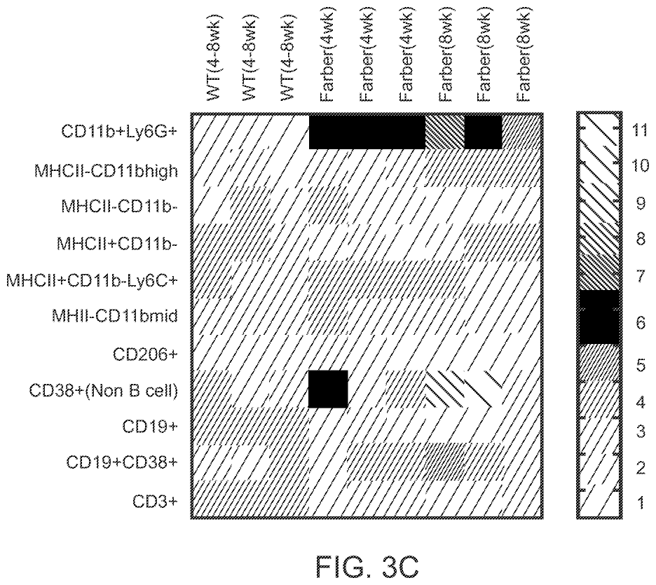

Example 3. Systemic (blood) immune cell populations. Results are depicted in FIG. 3. Inflammatory cell populations characteristic of an inflammatory state were analyzed from control 4 and 8 week old wild-type and Farber mouse blood samples. The population of Ly6G, CD11b double positive CD45⁺ neutrophils and CD11b⁺MHCII⁻CD45⁺ activated monocytes were determined from 7 week old Farber mice that were administered 10 mg/kg/dose RVT-801 once weekly beginning at 3 weeks of age for a total of 4 doses over 4 weeks. Heat map analysis of fold change differences in the frequency of immune cells in the blood of age-matched Farber mice and littermate controls (WT). Each column represents an individual animal. Fold change was calculated by setting the average WT value to 1.

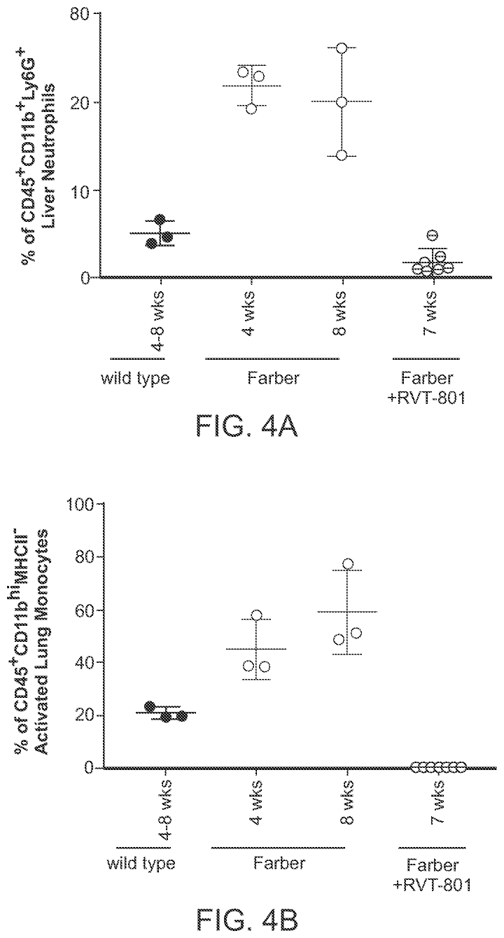

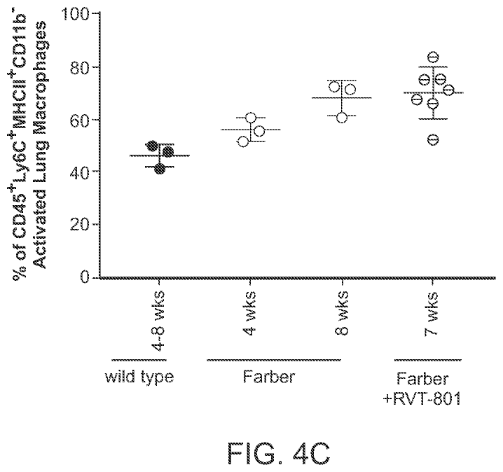

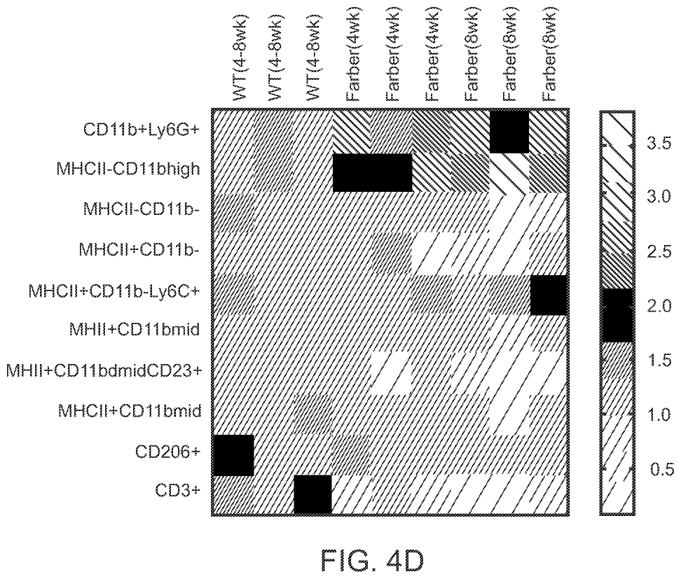

Example 4. Pulmonary immune cell populations. Results are depicted in FIG. 4. Inflammatory cell populations characteristic of an inflammatory state were analyzed from control 4 and 8 week old wild-type and Farber mouse lung tissue. The population of Ly6G, CD11b double positive CD45⁺ neutrophils and CD11b⁺MHCII⁻CD45⁺ activated monocytes were determined from 7 week old Farber mice that were administered 10 mg/kg/dose RVT-801 once weekly beginning at 3 weeks of age for a total of 4 doses over 4 weeks. Heat map analysis of fold change differences in the frequency of immune cells in the lung of age-matched Farber mice and littermate controls (WT). Each column represents an individual animal. Fold change was calculated by setting the average WT value to 1. Also reported in FIG. 4C is an additional macrophage population that is CD45+Ly6C-MHCII+CD11b−.

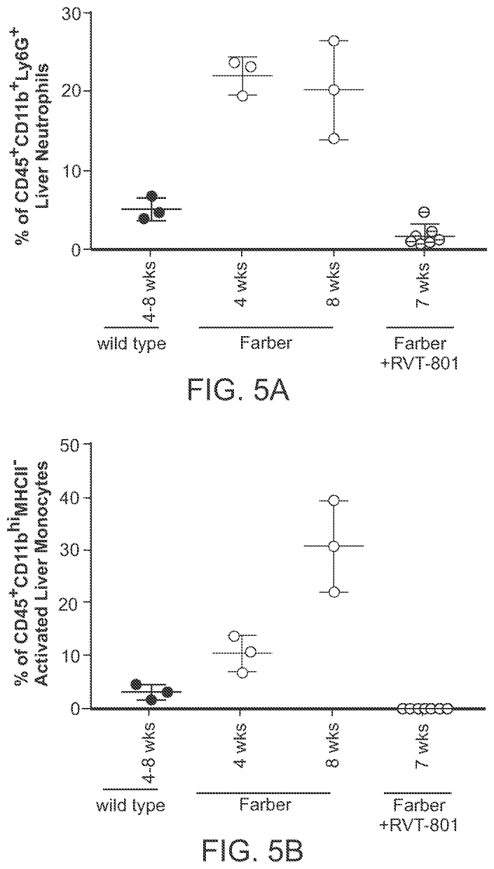

Example 5. Hepatic immune cell populations. Results are depicted in FIG. 5. Inflammatory cell populations characteristic of an inflammatory state were analyzed from control 4 and 8 week old wild-type and Farber mouse liver tissue. The population of Ly6G/CD11b double positive CD45⁺ neutrophils and CD11b^{+hi}MHCII−CD45⁺ activated monocytes were determined from 7 week old Farber mice that were administered 10 mg/kg/dose RVT-801 once weekly beginning at 3 weeks of age for a total of 4 doses over 4 weeks

Example 6 was performed in the following manner. Farber mice Asah1P36IR P36IR were administered recombinant human acid ceramidase (RVT-801) at a dose of 0 (saline vehicle, 0.1, 1, 3 and 10 mg/kg once weekly starting at three weeks of age fro six weeks. Ceramide and sphingosine were measured in tissue. MCP-1 was measured from plasma. Body and tissue weights were measured weekly and terminally. Histopathology of the brain, liver, lung, thyroid, trachea, esophagus, skeletal muscle, thymus, heart, spleen, kidney, femoro-tibial joint and femur (bone marrow).

A single intraperitoneal dose of 0, 0.1, 1, 3 and 10 mg/kg of recombinant human acid ceramidase (RVT-801) was administered to CD-1 mice at 3.5 weeks (mice were genotyped and weaned at 3-4 weeks). Pharmacokinetic and tissue distribution analysis of recombinant human acid ceramidase (RVT-801) was assessed at pre-dose, 0.5, 1, 2, 3, 4, 6, 8, 10, 12, 18, and 24 hours post-dose.

Farber mice that were dosed with 10 mg/kg RVT-801 for pharmacokinetic and tissue distribution studies were measured at two time points (n=3 at each time point), i.e., at 6 and 24 hours. Pharmacokinetics of RVT-801 in CD-1 and Farber mouse serum was determined by enzyme-linked immunosorbent assay (ELISA) at BioAgilytix.

Disposition of RVT-801 in liver, spleen, brain, kidney, heart and lung was determined by bespoke ELISA at BioAgilytix.

Bronchiole alveolar lavage fluid (BALF) was taken at 24 hours post-dose from CD-1 mice following a single dose of recombinant human acid ceramidase (RVT-801). Disposition of RVT-801 in BALF-derived macrophages, generated via centrifugation of BALF, was determined by bespoke ELISA by BioAgilytix.

Farber mice dosed with 10 mg/kg RVT-801 once-weekly for 4 doses (n=7 of each genotype: n=3 per age group) were studied by immunophenotyping. Flow samples were taken of whole blood, liver, spleen and lung.

Monocytes of the following types were recorded: CD11b, CD11c, CD23, CD45, CD68, CD86, CD206, CCR2, Ly6G, Ly6C, CX3CR1, and F4/80.

Lymphocytes of the following types were recorded: CD3, CD4, CD8, CD19, CD38, CD44, CD45, CD62L, CD127, CCR7, MHCII, CCR6, and CXCR3.

Example 6. As shown at 4× (FIG. 6A-C, J-L) and 20× (FIG. 6D-I, M-O) magnification in FIGS. 6A-O bone samples from wild type WT), control Farber mice and Farber mice dosed with RVT-801, were demineralized, fixed, and stained with hematoxylin and eosin (H&E) for histopathological assessment. A hallmark symptom of Farber disease is the formation of large, painful nodules at, or near the joints. Characteristic physis (growth plate) of consistent width, robust bony trabeculae and linearly organized chondrocytes within the primary spongiosa was observed in WT joint (FIG. 6A,D). Similar to Farber patients, control Farber mice exhibited soft tissue histiocyte infiltration, primarily of the femoro-tibial joint ligaments and adipose tissues (FIG. 6B,H). Physeal dysplasia showed uneven to nodular physeal thickening in control Farber mice, often with disrupted chondrocyte organization: where Farber mouse physis was thin and lacking primary spongiosa (FIG. 6B,E). All control Farber mice exhibited thinning of the bony trabeculae in the epiphysis and metaphysis and notable histiocytic infiltration compared to WT (FIG. 6B vs 6A). The physeal plate of the Farber mouse administered 10 mg/kg/dose RVT-801 once weekly for 6-weeks appeared organized with normal size diaphysis trabeculae and normal structural elements (FIG. 6C,F). Histiocyte accumulation in the femoro-tibial joint of control Farber mice was overwhelming the fat pad (FIG. 6G-H). Decreased histiocytes in the intra-articular soft tissue was evident in RVT-801 treated Farber mice, where fat tissue was preserved (FIG. 61). WT bone marrow was confluent and basophilic due to maturing hematopoietic cells (FIG. 6J,M). Accumulated pale foamy histiocytes were present within the bone marrow of all control Farber mice (FIG. 6K,N). Bone marrow infiltrates were not observed in Farber mice treated with 10 mg/kg RVT-801, consistent with WT bone marrow (FIG. 6L,O). Due to the nature of the tissue, immunological assessment of the bone and joint were not conducted.

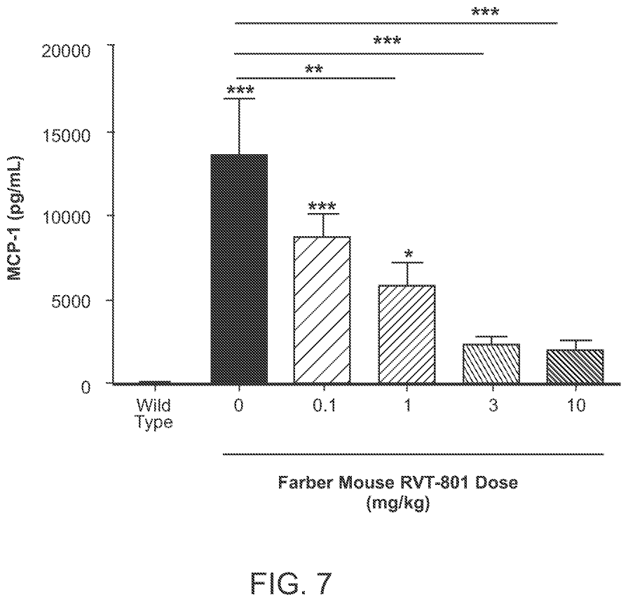

Example 7. FIG. 7A shows a dose-dependent reduction in elevated MCP-1 upon administration of recombinant human acid ceramidase (RVT-801) at 10 mg/kg/dose once-weekly to Farber mice for 6 weeks, which is not different from WT levels at ≥3 mg/kg/dose once-weekly.

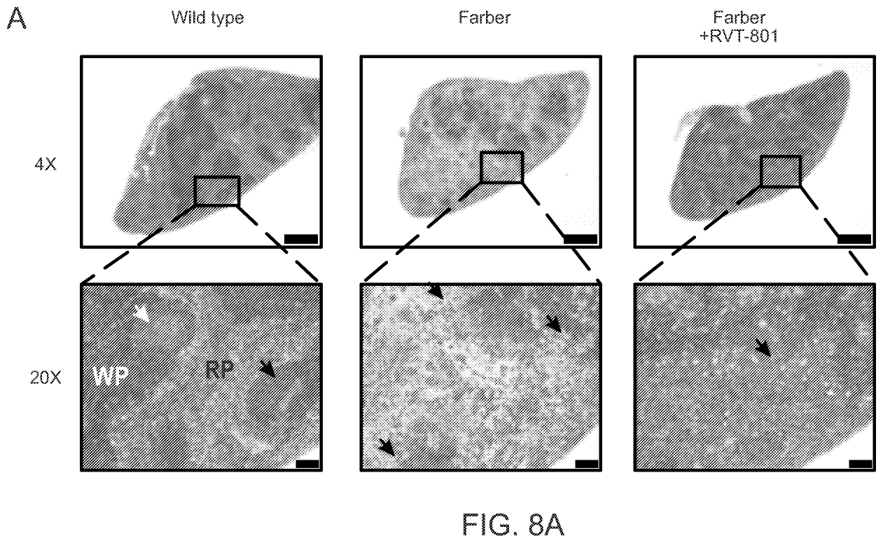

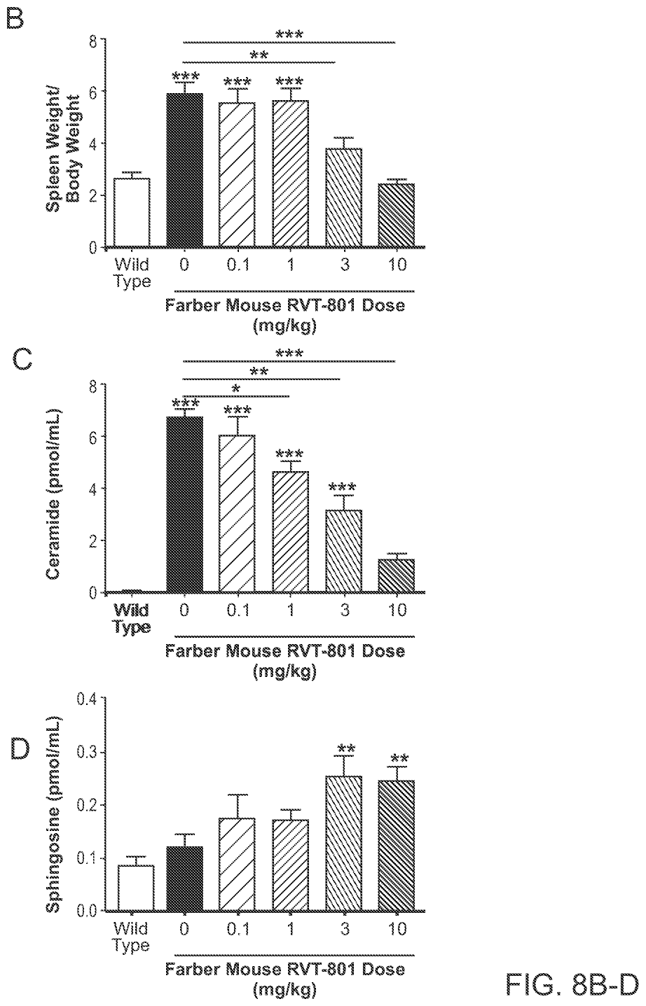

Example 8. Wild type mouse spleens showed typical ratios of red and white pulp with evident germinal center and lymphoid follicles (FIG. 8A, left panels-4× and 20× magnification). Control Farber mice spleens showed increased inflammation and histocyte infiltration with decreased white and red pulp (FIG. 8A, middle panels-4× and 20× magnification). RVT-801 at 0.1, and 1 mg/kg/week did not appear to reduce spleen Farber mouse pathology with extramedullary hematopoiesis (EMH). Farber mouse dosed with ≥3 mg/kg/week conserved spleen white pulp with robust germinal centers and decreased histiocytic infiltrates and EMH (FIG. 8A, right panels-4× and 20× magnification). RVT-801 at 10 mg/kg/week also ameliorated single cell necrosis, neutrophilic inflammation, and pigment (hemosiderin) red pulp.

Splenomegaly is associated with clinical Farber disease, and significantly higher control Farber mouse spleen to body weight (BW) ratios were significantly higher than WT (FIG. 8B). While absolute Farber mouse spleen weights showed no significant correlation to RVT-801 treatment, the BW-normalized spleen weights of Farber mice receiving≥3 mg/kg/week RVT-801 were resolved to not significantly different from WT (FIG. 8B).

FIG. 8C shows that control Farber mice had significantly higher splenic ceramide levels compared to WT, which dose-dependently decreased with increasing RVT-801 (0.1, 1, 3, or 10 mg/kg/dose once-weekly for 6 weeks); resulting ceramide levels following 10 mg/kg/dose were indistinguishable from WT (FIG. 8C).

FIG. 8D shows splenic sphingosine levels were not different between WT and control Farber mice, however, Farber mouse sphingosine levels significantly increased at ≥3 mg/kg/dose RVT-801.

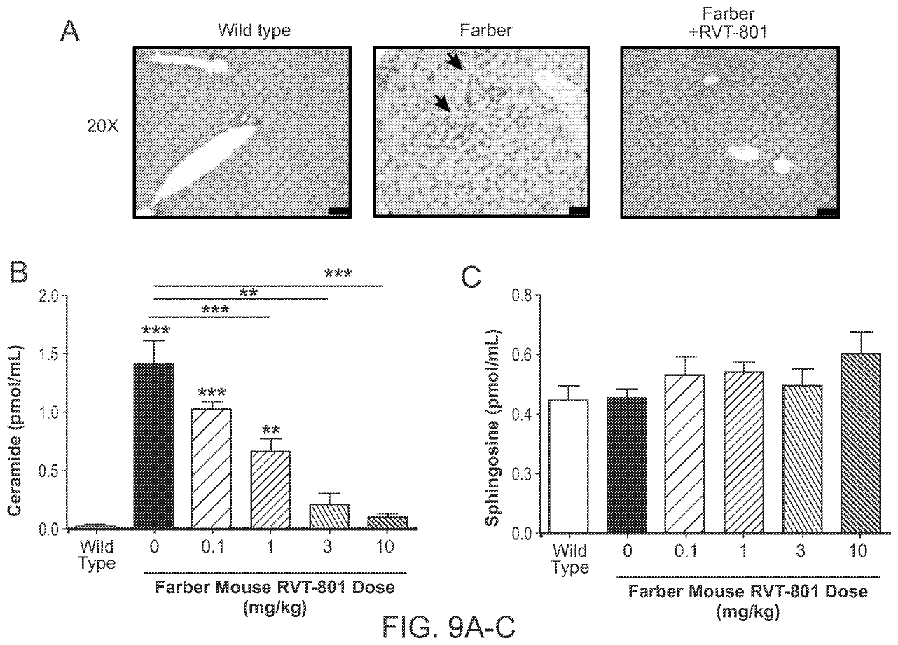

Example 9. Data from livers of wild type mice, Farber mice and Farber mice treated with RVT-801 is presented in FIGS. 9A-E. Histological evaluation of liver specimens for wild type (FIG. 9A, left panel), control Farber mice (FIG. 9A, middle panel) and Farber mice treated with 10 mg/kg/dose RVT-801 once weekly for 6 weeks are shown at 20× magnification. Novel hepatic thrombus formation in the vasculature primarily consisted of histiocytic aggregates without other features of true thrombi (FIG. 9A, middle panel). Histiocytic infiltration, mixed-cell inflammation, hepatocellular degeneration and necrosis, and thrombus formation decreased with increasing RVT-801 (FIG. 9A, right panel), with no observer thrombi observed at ≥3 mg/kg/week RVT-801. Control Farber mouse livers exhibited increased foamy histiocytes and contained significantly higher ceramide concentrations than WT as previously reported (He et al.) (FIG. 9A, middle panel and 9B)

FIG. 9B shows a significant increase in hepatic ceramide levels in control Farber mice when compared to wild-type mice, and a dose-dependent reduction in ceramide levels in livers of Farber mice dosed with 0.1, 1, 3 and 10 mg/kg RVT-801 once-weekly for 6 weeks, nearly approaching levels in wild type mice.

FIG. 9C shows increased sphingosine in a dose dependent manner in livers of Farber mice treated with RVT-801 compared to control Farber mice, though this change did not reach statistical significance.

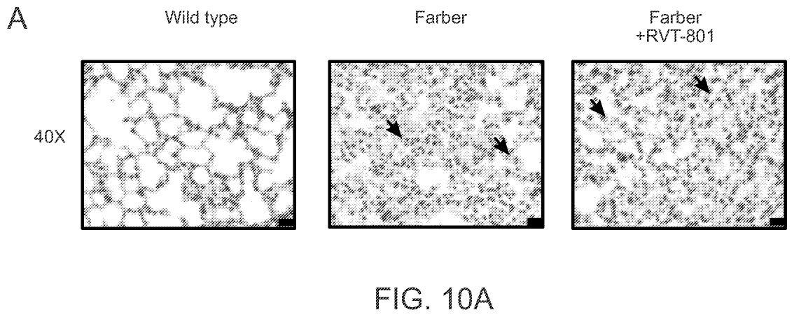

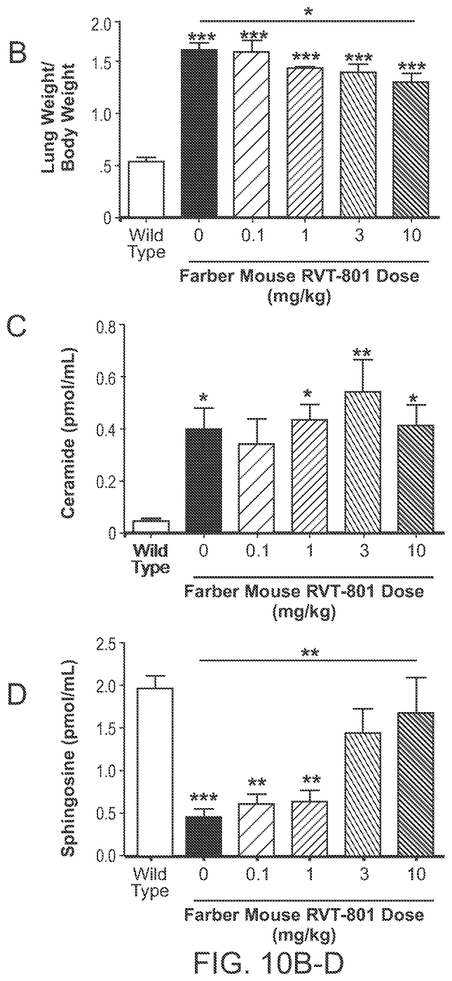

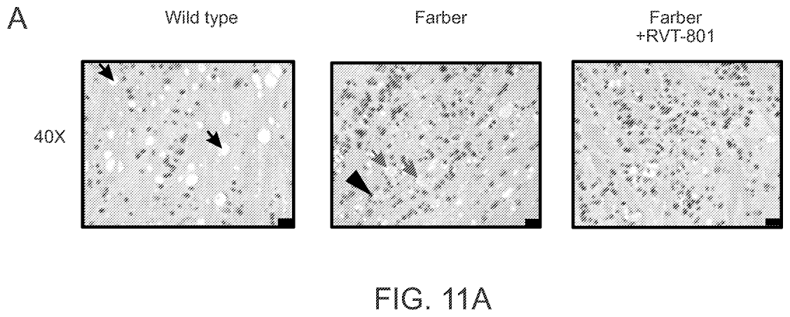

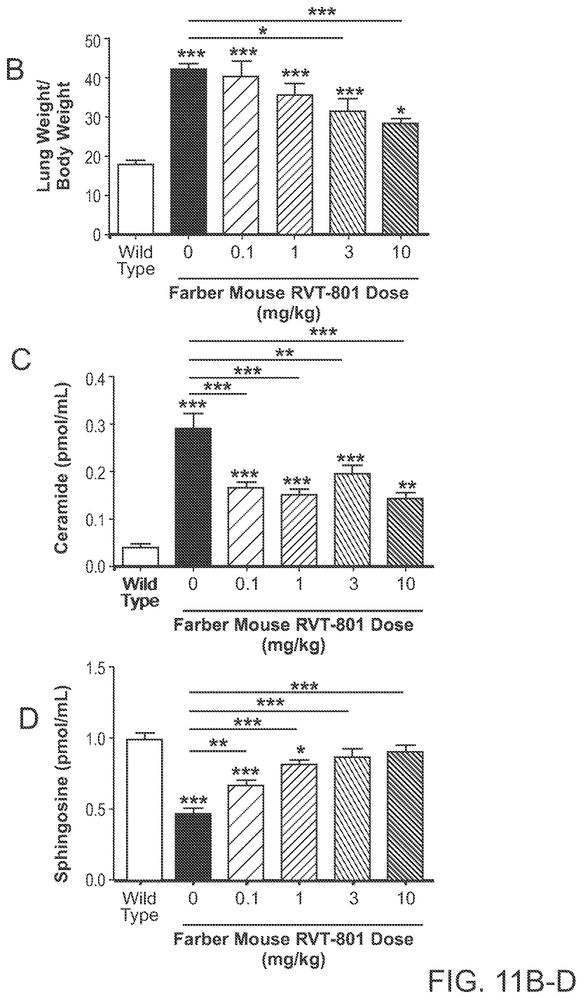

Example 10. Data on lungs of wild type mice, Farber mice and Farber mice treated with RVT-801 is presented in FIGS. 10A-C.

FIGS. 10A-C show images of lungs of wild type mice, control Farber mice and Farber mice treated with 10 mg/kg/dose RVT-801 once-weekly for 6 weeks. The image of wild type mouse lung shows patent alveolar sacs with tin cellular walls and no infiltrating cells (FIG. 10A, left panel), while the control Farber mouse lung shows reduced or collapsed alveolar sacs with inflamed cellular walls and cellular infiltrates. Alveolar proteinosis was present in all control Farber mice regardless of RVT-801 treatment (FIG. 10A, middle panel), with interstitial histiocyte infiltration frequently expanding alveolar septae that aggregated in alveolar spaces. RVT-801 dosed up to 10 mg/kg/dose once-weekly for 6 weeks appeared to have limited impact on the histopathology of the Farber mouse lung.

FIG. 10B shows a comparison of lung to body weights (BW) in wild type mice, control Farber mice and Farber mice treated with RVT-801. The mean weight of control WT and FM lungs were similar (not shown). Control Farber mice also had significantly higher lung to BW ratios than WT, however, FM treated with 10 mg/kg/week RVT-801 had significantly lower BW-normalized lung weights than untreated FM (FIG. 10B).

FIG. 10C shows lung ceramide levels in control Farber mice were significantly higher than WT, and treatment with RVT-801 did not appear to impact lung ceramide at any dose (FIG. 10C).

FIG. 10C showed little to no effect on ceramide levels in lungs of Farber mice treated with RVT-801, whereas FIG. 10D shows a dose-related increase in sphingosine levels in lungs of Farber mice treated with RVT-801, comparable to level noted in wild-type mice at the 10 mg/kg dose.

However, FIG. 10D shows lung sphingosine levels in control Farber mice were significantly lower than in WT: importantly, sphingosine levels increased in a dose-dependent manner and were statistically no different from WT at ≥3 mg/kg/week (FIG. 10D).

Example 11. Data on brains of wild type mice, control Farber mice and Farber mice treated with RVT-801 is presented in FIGS. 11A-C. FIG. 11A depicts brain specimens from wild type mice, control Farber mice and Farber mice treated with RVT-801. Glial necrosis occurred throughout the white matter of the forebrain, midbrain, and hindbrain in control Farber mice and was highly associated with areas of histiocytic infiltrates, which did not appear to be affected by RVT-801 treatment (FIG. 11A).

FIG. 11B shows control Farber mouse BW-normalized brain weights were approximately twice those of WT. Weekly RVT-801 doses of ≥3 mg/kg resulted in significantly decreased BW-normalized brain weights relative to untreated FM (FIG. 11B). Absolute FM brain weights were significantly lower than those of WT and generally increased with dose and were not significantly different from WT absolute brain weight at ≥1 mg/kg/week (not shown).

FIG. 11C shows a significant increase in brain ceramide levels in control Farber mice when compared to wild type brain ceramide levels, and shows a consistent decrease in ceramide levels in brains of Farber mice treated with RVT-801 at all doses. FIG. 11D shows a significant decrease in brain sphingosine levels in control Farber mice when compared to wild type brain sphingosine levels, and a dose-related increase in sphingosine levels in brains of Farber mice treated with RVT-801 that is not different from WT at ≥3 mg/kg/dose once-weekly for 6 weeks.

The foregoing embodiments and advantages are merely exemplary and are not to be construed as limiting the present invention. The present teachings can be readily applied to other types of apparatuses, experiments and surgical procedures. Also, the description of the embodiments of the present invention is intended to be illustrative and not to limit the scope of the claims. Many alternatives, modifications, and variations will be apparent to those skilled in the art.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

References discussed in the application, which are incorporated by reference in their entirety, for their intended purpose, which is clear based upon its context.

The disclosures of each and every patent, patent application, publication, and accession number cited herein are hereby incorporated herein by reference in their entirety.

While present disclosure has been disclosed with reference to various embodiments, it is apparent that other embodiments and variations of these may be devised by others skilled in the art without departing from the true spirit and scope of the disclosure. The appended claims are intended to be construed to include all such embodiments and equivalent variations.

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.
Ausubel, F. M., 1999, Short Protocols in Molecular Biology (Wiley 1999).
Bauer, et al. (inventor), Cornel Research Foundation, Inc. (assignee), 2003 Jul. 22, published as U.S. Pat. No. 6,596,509.
Farber, S. (1952) A lipid metabolic disorder—disseminated “Lipogranulomatosis”—a syndrome with similarity to, and important difference from, Niemann-Pick and Hand-Schuller-Christian disease. Am. J. Dis. Child, 84:499.
Koch et al., 1996, Molecular Cloning and Characterization of a Full-Length Complementary DNA Encoding Human Acid Ceramidase, J. Biol. Chem. 271:33110-33115
Solyom A, Huegle B, Magnusson B, Makay B, Arslan N, Mitchell J, Tanpaiboon P, Guelbert N, Puri R, Jung L, Grigelioniene G, Ehlert K, Beck M, Simonaro C, and Schuchman E., 2015, Farber Disease: Important Differential Diagnostic Information for JIA and Other Inflammatory Arthritis Phenotypes Is Revealed By Data from the Largest Clinical Cohort to Date,” Arthritis Rheumatol. 2015:67 (suppl 10), abstract
Lampe C, Bellettato C, Karabul N, and Scarpa M., 2013, Mucopolysaccharidoses and other lysosomal storage diseases. Rheum Dis Clin N Am. May: (39) (2): 431-55.
Hugle B, Mueller L, Levade T., 2014. Why Farber disease may be misdiagnosed as juvenile idiopathic arthritis. The Rheumatologist, June 1.
Dworski, S., Lu, P., Khan, A., Maranda, B., Mitchell, J., Tanpaiboon, P., & +22 additional authors (2016). Acid Ceramidase Deficiency is characterized by a unique plasma cytokine and ceramide profile that is altered by therapy. Biochimica et Biophysica Acta. 1863 (2).
Alayoubi, A. M., J. C. Wang, B. C. Au, S. Carpentier, V. Garcia, S. Dworski, S. El-Ghamrasni, K. N. Kirouac, M. J. Exertier, Z. J. Xiong, G. G. Prive, C. M. Simonaro, J. Casas, G. Fabrias, E. H. Schuchman, P. V. Turner, R. Hakem, T. Levade, and J. A. Medin (2013). Systemic ceramide accumulation leads to severe and varied pathological consequences. EMBO Mol. Med, 5:827-842.
Bae, J. S., Jang, K. H., Schuchman, E. H., and Jin, H. K., 2004, Comparative effects of recombinant acid sphingomyelinase administration by different routes in Niemann-Pick disease mice. Exp Anim, 53:417-421.
Becker, K. A., Riethmüller, J., Lüth, A., Döring, G., Kleuser, B., and Gulbins, E. (2010). Acid Sphingomyelinase Inhibitors Normalize Pulmonary Ceramide and Inflammation in Cystic Fibrosis. Am. J. Respir. Cell. Mol. Biol., 42 (6): 716-724.
Beckmann, N., et al., 2018, Pathological manifestations of Farber disease in a new mouse model, Biol. Chem., 399 (10): 1183-1202. Bernardo et al., 1995, Purification, characterization, and biosynthesis of human acid ceramidase, J. Biol. Chem. 270:11098-102.1.
Boado, R. J., Lu, J. Z., Hui, E. K., Lin, H., and Pardridge, W. M. (2016). Insulin receptor antibody-alpha-N-acetylglucosaminidase fusion protein penetrates the primate blood-brain barrier and reduces glycosaminoglycans in Sanfillippo type B fibroblasts. Mol.
Pharm., 13:1385-92.
Chatelut, M., Harzer, K., Christomanou, H., Feunteun, J., Pieraggi, M. T., Paton, B. C., Kishimoto, Y., O'Brien, J. S., Basile, J. P., Thiers, J. C., Salvayre, R., and Levade, T. (1997). Model SV40-transformed fibroblast lines for metabolic studies of human prosaposin and acid ceramidase deficiencies. Clin Chim Acta, 262:61-76.
Cohen and Boyer (inventors), Board of Trustees of the Leland Stanford Jr. University (assignee), Process for producing biologically functional molecular chimeras, published as U.S. Pat. No. 4,237,224.
Desnick, R. J. and Schuchman, E. H. (2012). Enzyme replacement therapy for lysosomal storage diseases: lessons from 20 years of experience and remaining challenges. Annu Rev Genomics Hum Genet, 13:307-335.
Dworski, S., Berger, A., Furlonger, C., Moreau, J. M., Yoshimitsu, M., Trentadue, J., Au, B. C., Paige, C. J., and Medin, J. A., 2015, Markedly perturbed hematopoiesis in acid ceramidase deficient mice. Haematologica, 100 (5): e162-165.
Dworski, S., Lu, P., Khan, A., Maranda, B., Mitchell, J. J., Parini, R., Di Rocco, M., Hugle, B., Yoshimitsu, M., Magnusson, B., Makay, B., Arslan, N., Guelbert, N., Ehlert, K., Jarisch, A., Gardner-Medwin, J., Dagher, R., Terreri, M. T., Lorenco, C. M., Barillas-Arias, L., Tanpaiboon, P., Solyom, A., Norris, J. S., He, X., Schuchman, E. H., Levade, T., and Medin, J. A., 2017, Acid Ceramidase Deficiency is characterized by a unique plasma cytokine and ceramide profile that is altered by therapy. Biochim Biophys Acta, 1863 (2): 386-394 (E-pub December 2016. Eliyahu, E., Park, J. H., Shtraizent, N., He, X., and Schuchman, E. H. (2007). Acid ceramidase is a novel factor required for early embryo survival. FASEB J., 21:1403-9.
Dzau, V. J, and Kaneda, Yasufumi (inventors), Method for producing in vivo delivery of therapeutic agents via liposomes, published as U.S. Pat. No. 5,631,237.
Ekwuribe, N. N. (inventor), Protein Delivery, Inc. (assignee), 1997 Oct. 28, Conjugation-stabilized therapeutic agent compositions, delivery and diagnostic formulations comprising same, and method of making and using the same, published as U.S. Pat. No. 5,681,811.
Eliyahu, E., N. Shtraizent, K. Martinuzzi, J. Barritt, X. He, H. Wei, S. Chaubal, A. B. Copperman, and E. H. Schuchman, 2010, Acid ceramidase improves the quality of oocytes and embryos and the outcome of in vitro fertilization. FASEB J. 24:1229-1238.
Eliyahu, E., N. Shtraizent, R. Shalgi and E. H. Schuchman, 2012, Construction of conditional acid ceramidase knockout mice and in vivo effects on oocyte development and fertility. Cellular physiology and biochemistry, Int'l J Exp. Cellular Phys., Bioch. and Pharmacol., 30:735-748.
Ferlinz et al., 2001, Human acid ceramidase: processing, glycosylation, and lysosomal targeting, J. Biol. Chem., 276 (38): 35352-60).
Frohbergh, M. E., Guevara, J. M., Greisamer, R. P., Barbe, M. F., He, X., Simonaro, C. M., and Schuchman, E. H. (2016). Acid ceramidase treatment enhances the outcome of autologous chondrocyte implantation in a rat osteochondral defect model. Osteoarthritis Cartilage, 24:752-762.
Gatt, S. (1963). Enzymic hydrolysis and synthesis of ceramides. J Biol Chem, 238:3131-3133.
He et al., 2003, Purification and characterization of recombinant, human acid ceramidase, J. Biol. Chem. 278:32978-32986.
Hollak, C. E. and Wijburg, F. A. (2014). Treatment of lysosomal storage disorders: successes and challenges. J Inherit Metab Dis, 37:587-598.
Hsu C. C. (inventor), Genentech, Inc. (assignee), 1997 Aug. 5. Method for preparing liposomes, published as U.S. Pat. No. 5,653,996.
Lee, K.-D., et al. (inventors), President and Fellws of Harvard College and the University of Pennsylvania, Intracellular delivery of macromolecules, published as U.S. Pat. No. 5,643,599.
Jablonski, K. A., Amici, S. A., Webb, L. M., Ruiz-Rosado, Jd. D., Popovich, P. G., Partida-Sanchez, S., and Guerau-de-Arellano, M., 2015, Novel Markers to Delineate Murine M1 and M2 Macrophages. PLOS ONE 10 (12): e0145342.
Koch et al., 1996, Molecular Cloning and Characterization of a Full-Length Complementary DNA Encoding Human Acid Ceramidase. J. Biol. Chem. 271:33110-33115
LeFourm V., et al. (inventors), Selexis S. A. (applicant), 2014 Aug. 2, Enhanced Transgene Expression And Processing, International application under the Patent Cooperation Treaty, published as WO2014/118619.
Li, C. M., J. H. Park, X. He, B. Levy, F. Chen, K. Arai, D. A. Adler, C. M. Disteche, J. Koch, K. Sandhoff, and E. H. Schuchman, 1999, The human acid ceramidase gene (asah): Structure, chromosomal location, mutation analysis, and expression. Genomics, 62 (2): 223-231.
Li, C. M., S. B. Hong, G. Kopal, X. He, T. Linke, W. S. Hou, J. Koch, S. Gatt, K. Sandhoff, and E. H. Schuchman, 1998, Cloning and characterization of the full-length cDNA and genomic sequences encoding murine acid ceramidase. Genomics, 50 (2): 267-274.
Li, C. M., J. H. Park, C. M. Simonaro, X. He, R. E. Gordon, A. H. Friedman, D. Ehleiter, F. Paris, K. Manova, S. Hepbildikler, Z. Fuks, K. Sandhoff, R. Kolesnick, and E. H. Schuchman, 2002, Insertional mutagenesis of the mouse acid ceramidase gene leads to early embryonic lethality in homozygotes and progressive lipid storage disease in heterozygotes. Genomics, 79 (2): 218-224.
Murray J M, Thompson, A M, Vitsky A, Hawes M, Chuang W L, Pacheco J, Wilson S, McPherson J M, Thurberg B L, Karey K P, and Andrews L., 2015, Nonclinical safety assessment of recombinant human acid sphingomyelinase (rhASM) for the treatment of acid sphingomyelinase deficiency: the utility of animal models of disease in the toxicologyevaluation of potential therapeutics. Mol Genet Metab. 114:217-225.
Nikolova-Karakashian et al., 2000, Ceramidases, Methods Enzymol. 311:194-201.
Hassler et al., 1993, Ceramidases: enzymology and metabolic roles, Adv. Lipid Res. 26:49-57.
Okino, N., He, X., S. Gatt, K. Sandhoff, M. Ito, and E. H. Schuchman, 2003, The reverse activity of human acid ceramidase. J Biol Chem, 278 (32): 29948-29953.
Park and Schuchman, 2006, Acid ceramidase and human disease, Biochim. Biophys.
Acta., 1758 (12): 2133-2138.
Realini, N., Palese, F., Pizzirani, D., Pontis, S., Basit, A., Bach, A., Ganesan, A., and Piomelli, D. (2015). Acid ceramidase in melanoma: expression, localization and effects of pharmacological inhibition. J Biol Chem, N291: 2422-2434.
Roberts and Lauer, 1979, Maximizing gene expression on a plasmid using recombination in vitro, Methods in Enzymology 68:473-82.
Roh, J. L., Park, J. Y., Kim, E. H., and Jang, H. J., 2016, Targeting acid ceramidase sensitises head and neck cancer to cisplatin. Eur J Cancer, 52:163-72.
Sambrook, J., et al., Molecular Cloning: A laboratory manual (Cold Springs Harbor 1989.
Shiffmann, S., Hartmann, D., Birod, K., Ferreiros, N., Schreiber, Y., Zivkovic, A., Geisslinger, G., Grösch, S., and Stark, H., 2012, Inhibitors of Specific Ceramide Synthases. Biochimie, 94 (2): 558-565.
Schuchman, E. H. (inventor), Icahn School of Medicine at Mount Sinai (applicant), 2016 Feb. 11, Therapeutic Acid Ceramidase Compositions And Methods Of Making And Using Them, published as U.S. Published Patent Application No. US 2016/0038574 A1.
Schuchman, E. (inventor), Icahn School of Medicine at Mount Sinai (applicant), 2018 Jan. 12, Compositions and Methods for Treating Farber Disease, International application under the Patent Cooperation Treaty, WO 2018/132667.
Schuchman, E. H. (inventor), Icahn School of Medicine at Mount Sinai (applicant), Recombinant Human Acid Ceramidase Production Process. International application under the Patent Cooperation Treaty, PCT/US2018/052463 (not yet published).
Schuchman, E. H., 2016, Acid ceramidase and the treatment of ceramide diseases. The expanding role of enzyme replacement therapy. Biochim Bipphys Acta, 1862:1459-1471.
Simonaro, C. M., Sachot, S., Ge, Y., He, X., DeAngelis, V. A., Eliyahu, E., Leong, D. J., Sun, H. B., Mason, J. B., Haskins, M. E., Richardson, D. W., and Schuchman, E. H., 2013, Acid ceramidase maintains the chondrogenic phenotype of expanded primary chondrocytes and improves the chondrogenic differentiation of bone marrow-derived mesenchymal stem cells. PLOS One, 8: e62715.
Shtraizent, N., E. Eliyahu, J. H. Park, X. He, R. Shalgi and E. H. Schuchman, 2008, Autoproteolytic cleavage and activation of human acid ceramidase. J Biol Chem, 283 (17): 11253-11259. Sugita, M., Dulaney, J. T., and Moser, H W (1972). Ceramidase deficiency in Farber's disease (lipogranulomatosis). Science, 178 (4065): 1100-1102.
Sugita, M., Dulaney, J. T., and Moser, H. W., 1972, Ceramidase deficiency in Farber's disease (lipogranulomatosis). Science, 178 (4065): 1100-1102.
Teichgräber, V. et al., 2008, Ceramide accumulation mediates inflammation, cell death and infection susceptibility in cystic fibrosis, Nature Medicine 14:382-391.
Wang et al., 1997, pH-sensitive immunoliposomes mediate target-cell-specific delivery and controlled expression of a foreign gene in mouse, Proc. Nat'l Acad. Sci. USA 84:7851-5.
Wolff et al., 1984, The use of monoclonal anti-Thy 1 IgG1 for the targeting of liposomes to AKR-A cells in vitro and in vivo, Biochim. Biophys. Acta 802:259-73.
Bangham, A. D., et al., 1965, Diffusion of univalent ions across the lamellae of swollen phospholipids, J. Mol. Biol. 13:238-52.
Young et al., 2013, Sphingolipids: regulators of crosstalk between apoptosis and autophagy, J. Lipid. Res. 54:5-19.
Yu, Fabian P. S., et al. 2017, Chronic lung injury and impaired pulmonary function in a mouse model of acid ceramidase deficiency, Lung Cellular and Mol. Physiol. 314 (3): L406-L420.
Zeidan et al., 2008, Molecular targeting of acid ceramidase: implications to cancer therapy, Curr. Drug Targets, 9 (8): 653-661.

1. A method for treating inflammation associated with Farber disease in a subject in need thereof, the method comprising administering to the subject a pharmaceutical composition comprising a recombinant human acid ceramidase in a therapeutically effective amount of about 0.1 mg/kg to about 50 mg/kg.

2. The method according to claim 1, wherein the recombinant acid ceramidase comprises UniProt Q13510, UniProt Q9H715, UniProt Q96AS2, OMIM 228000, NCBI Gene 427, NCBI RefSeq NP_808592, NCBI RefSeq NP_004306, NCBI RefSeq NM_177924, NCBI RefSeq NM_004315, NCBI UniGene 427, NCBI Accession Q13510, NCBI Accession AAC73009, or a combination thereof.

3. The method according to claim 1, wherein the recombinant human acid ceramidase is a rhAC encoded by the ASAH1 gene (NCBI UniGene GeneID No. 427).

4. The method according to claim 1, wherein the recombinant human acid ceramidase comprises the sequence of SEQ ID NO: 1.

5. The method according to claim 1, wherein the recombinant acid ceramidase comprises the sequence of UniProt Q 13510.

6. The method according to claim 1, wherein the recombinant acid ceramidase comprises the sequence of NCBI RefSeq NP_808592.

7. The method according to claim 1, further comprising administering a second anti-inflammatory agent in a therapeutically effective amount in combination with the recombinant human acid ceramidase.

8. The method according to claim 7, wherein the second anti-inflammatory agent is selected from the group wherein the second anti-inflammatory agent is selected from the group consisting of aceclofenac, alclofenac, amfenac, aminophenazone, ampiroxicam, ampyrone, amtolmetin guacil, anitrazafen azapropazone, bendazac, benzydamine, bromfenac, bumadizone, carprofen, celecoxib, cimicoxib, clofezone, clonixin, copper ibuprofenate, COX-inhibiting nitric oxide donator, deracoxib, dexibuprofen, dexketoprofen, diclofenac, diclofenac/misoprostol, diflunisal, droxicam, epirizole, ethenzamide, etodolac, etofenamate, etoricoxib, famprofazone, felbinac, fenamic acid, fenbufen, fenclofenac, fenclozic acid, fenoprofen, feprazone, firocoxib, floctafenine, flumizole, flunixin, fluproquazone, flurbiprofen, ibuprofen, indomethacin, indometacin famesil, indoprofen, ketoprofen, ketorolac, licofelone, lonazolac, lomoxicam, loxoprofen, lumiracoxib, magnesium salicylate, mavacoxib, mefenamic acid, meloxicam, meseclazone, miroprofen, mofebutazone, morazone, nabumetone, naproxcinod, naproxen, nepafenac, nimesulide, NOSH-aspirin, NS-398, oxaprozin, oxicam, oxyphenbutazone, parecoxib, phenazone, phenylbutazone, piroxicam, pirprofen, pranoprofen, proglumetacin, robenacoxib, rofecoxib, salicylic acid, salsalate, sulindac, suprofen, tarenflurbil, tenidap, tenoxicam, tepoxalin, tiaprofenic acid, tocilizumab, tolfenamic acid, tolmetin, valdecoxib, vedaprofen, and zomepirac.

9. The method according to claim 1, wherein the pharmaceutical composition is in solid or liquid form.

10. The method according to claim 9, wherein the liquid form is in a sterile injectable solution.

11. The method according to claim 9, wherein the liquid form is a sterile dispersion.

12. The method according to claim 1, wherein the pharmaceutical composition is in the form selected from the group consisting of tablets, capsules, elixirs, suspensions, a solution, a dispersion, and syrups.

13. The method according to claim 1, wherein the pharmaceutical composition further comprises one or more of the following: a binder, an excipient, a disintegrating agent, a lubricant, a sweetening agent, or a liquid carrier.

14. The method according to claim 1, wherein the pharmaceutical composition comprises saline or water.

15. A method for treating inflammation in tissues and organs of a subject in need thereof having Farber disease, comprising administering to the subject a therapeutically effective amount of a recombinant human acid ceramidase to inhibit or reduce pro-inflammatory potential of neutrophils and/or monocytes in the subject.

16. The method according to claim 15, wherein the recombinant acid ceramidase comprises UniProt Q13510, UniProt Q9H715, UniProt Q96AS2, OMIM 228000, NCBI Gene 427, NCBI RefSeq NP_808592, NCBI RefSeq NP_004306, NCBI RefSeq NM_177924, NCBI RefSeq NM_004315, NCBI UniGene 427, NCBI Accession Q13510, NCBI Accession AAC73009, or a combination thereof.

17. The method according to claim 15, wherein the recombinant human acid ceramidase is a rhAC encoded by the ASAH1 gene (NCBI UniGene GeneID No. 427).

18. The method according to claim 15, wherein the recombinant human acid ceramidase comprises the sequence of SEQ ID NO: 1.

19. The method according to claim 15, wherein the recombinant acid ceramidase comprises UniProt Q 13510.

20. The method according to claim 15, wherein the recombinant acid ceramidase comprises NCBI RefSeq NP_808592.

21.-43. (canceled)
